SEQUENCE LISTING (27 sequences)

SEQ ID NO: 1 - DNA, 383 bp, Homo sapiens
  exon (1)..(383)
tgggttccaggttccactggtgacgacatccagttgacccagtctccaggcaccctgtctttgtctccaggggaaagagccaccctctcctgcagggccagtcagagtgtcagcagcagctacttagcctggtaccagcagaagcctggccaggctcccaggctcctcatttatggtatatctagtagggccactggcatcccagacaggttcagtggcagtgtgtctgggacagacttcactctcaccatcagcagactggagcctgaagattttgcagtgtattactgtcatcagttcggtattttaccgtggacgttcggccaagggaccaaggtggaaatcaaaggaactgtggctgcaccatctgtcttcatcttccc

SEQ ID NO: 2 - DNA, 386 bp, Homo sapiens
  exon (1)..(386)
tgggttccaggttccactggtgacgaaattgtgttgacacagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagctggagatcaaacgaactgtggctgcaccatctgtcttcatcttccc

SEQ ID NO: 3 - DNA, 383 bp, Homo sapiens
  exon (1)..(383)
tgggttccaggttccactggtgacgaaattgtgttgacacagtctccaggcaccctgtctttgtctccaggggaaagagccaccctctcctgcagggccagtcagagtgtcaacagcagctacttagcctggtaccagcagaagcctggccaggctcccaggctcctcatttatggtacatctagtagggccactggcatcccagacaggttcagtggcagtgtgtctgggacagacttcactctcaccatcagcagactggagcctgaagattttgcagtgtattactgtcatcagttcggtattttaccgtggacgttcggccaagggaccaaggtggaaatcaaaggaactgtggctgcaccatctgtcttcatcttccc

SEQ ID NO: 4 - DNA, 386 bp, Homo sapiens
  exon (1)..(386)
tgggttccaggttccactggtgacgaaattgtgctgacacagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagctggagaccaaacgaactgtggctgcaccatctgtcttcatcttccc

SEQ ID NO: 5 - DNA, 383 bp, Homo sapiens
  exon (1)..(383)
tgggttccaggttccactggtgacgacatccagatgacccagtctccaggcaccctgtctttgtctccaggggaaagagccaccctctcctgcagggccagtcagagtgtcagcagcagctacttagcctggtaccagcagaagcctggccaggctcccaggctcctcatttatggtacatctagtagggccactggcatcccagacaggttcagtggcagtgtgtctgggacagacttcactctcaccatcagcagactggagcctgaagattttgcagtgtattactgtcatcagttcggtattttaccgtggacgttcggccaagggaccaaggtggaaatcaaaggaactgtggctgcaccatctgtcttcatcttccc

SEQ ID NO: 6 - DNA, 386 bp, Homo sapiens
  exon (1)..(386)
  misc_feature (30)..(30): n is a, c, g, or t
tgggttccaggttccactggtgacgaaatngtgatgacacagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagctggagatcaaacgaactgtggctgcaccatctgtcttcatcttccc

SEQ ID NO: 7 - DNA, 380 bp, Homo sapiens
  exon (1)..(380)
tgggttccaggttccactggtgacgaaatccagatgactcagtctccagccaccgtgtctgtgtctccgggggggagagccactctctcctgcagggccagtcagagtgttaacggtaacttagcctggtaccagcaaaaacctggccaggcccccaggctcctcatctatggtgcatccaccagggccgctggtgtcccagccaggttcagtggcagtgggtctgggacagagttcactctcaccatcagcagcctgcagtctgaagatattgcagtttattactgtcagcactataatgtcgtgctgcggacgttcggccaggggaccaaggtggagatcaaacgaactgtggctgcaccatctgtcttcatcttccc

SEQ ID NO: 8 - DNA, 380 bp, Homo sapiens; identical to SEQ ID NO: 7
  exon (1)..(380)

SEQ ID NO: 9 - DNA, 380 bp, Homo sapiens
  exon (1)..(380)
tgggttccaggttccactggtgacgacatccagttgacccagtctccagccaccgtgtctgtgtctccgggggggagagccactctctcctgcagggccagtcagagtgttaacggtaacttagcctggtaccagcaaaaacctggccaggcccccaggctcctcatctatggtgcatccaccagggccgctggtgtcccagccaggttcagtggcagtgggtctgggacagagttcactctcaccatcagcagcctgcagtctgaagatattgcagtttattactgtcagcactataatgtcgtgctgcggacgttcggccaggggaccaaggtggagatcaaacgaactgtggctgcaccatctgtcttcatcttccc

SEQ ID NO: 10 - DNA, 380 bp, Homo sapiens
  exon (1)..(380)
tgggttccaggttccactggtgacgaaacgacactcacgcagtctccagccaccgtgtctgtgtctccgggggggagagccactctctcctgcagggccagtcagagtgttaacggtaacttagcctggtaccagcaaaaacctggccaggcccccaggctcctcatctatggtgcatccaccagggccgctggtgtcccagccaggttcagtggcagtgggtctgggacagagttcactctcaccatcagcagcctgcagtctgaagatattgcagtttattactgtcagcactataatgtcgtgctgcggacgttcggccaggggaccaaggtggagatcaaacgaactgtggctgcaccatctgtcttcatcttccc

SEQ ID NO: 11 - DNA, 380 bp, Homo sapiens
  exon (1)..(380)
tgggttccaggttccactggtgacgaaattgtgctgacacagtctccagccaccgtgtctgtgtctccgggggggagagccactctctcctgcagggccagtcagagtgttaacggtaacttagcctggtaccagcaaaaacctggccaggcccccaggctcctcatctatggtgcatccaccagggccgctggtgtcccagccaggttcagtggcagtgggtctgggacagagttcactctcaccatcagcagcctgcagtctgaagatattgcagtttattactgtcagcactataatgtcgtgctgcggacgttcggccaggggaccaaggtggagatcaaacgaactgtggctgcaccatctgtcttcatcttccc

SEQ ID NO: 12 - DNA, 380 bp, Homo sapiens
  exon (1)..(380)
tgggttccaggttccactggtgacgacattgtgctgacccagtctccagccaccgtgtctgtgtctccgggggggagagccactctctcctgcagggccagtcagagtgttaacggtaacttagcctggtaccagcaaaaacctggccaggcccccaggctcctcatctatggtgcatccaccagggccgctggtgtcccagccaggttcagtggcagtgggtctgggacagagttcactctcaccatcagcagcctgcagtctgaagatattgcagtttattactgtcagcactataatgtcgtgctgcggacgttcggccaggggaccaaggtggagatcaaacgaactgtggctgcaccatctgtcttcatcttccc

SEQ ID NO: 13 - DNA, 386 bp, Homo sapiens
  exon (1)..(386)
tgggttccaggttccactggtgacgacatcgtgatgacccagtctccacgcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagctggagatcaaacgaactgtggctgcaccatctgtcttcatcttccc

SEQ ID NO: 14 - DNA, 386 bp, Homo sapiens
  exon (1)..(386)
tgggttccaggttccactggtgacgaaattgtgttgacacagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggtccagtggcagtgggtctgggacagacttcactctcaccatcagcagactggagcctgaagattttgcagtgtattactgtcagcagtatggtagctcacctccgtacacttttggccaggggaccaagctggagatcaaacgatctgtggctgcaccatctgtcttcatcttccc

SEQ ID NO: 15 - DNA, 386 bp, Homo sapiens
  exon (1)..(386)
tgggttccaggttccactggtgacgacatccagatgacccagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagctggagatcaaacgaactgtggctgcaccatctgtcttcatcttccc

SEQ ID NO: 16 - DNA, 386 bp, Homo sapiens
  exon (1)..(386)
tgggttccaggttccactggtgacgaaattgtgctgactcagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagctggagatcaaacgaactgtggctgcaccatctgtcttcatcttccc

SEQ ID NO: 17 - DNA, 386 bp, Homo sapiens
  exon (1)..(386)
tgggttccaggttccactggtgacgaaattgtgctgacacagtctccaggcaccctgtctttgtctccaggggaaagagccaccctctcctgcagggccagtcaaagtattagcagcagcttcttagcctggtaccagcagaaacctggccagactcccaggctcctcatctatggtgcatccaacagggccactggcatcccagacaggttcagtggcagtgggtctgggacagacttcactctcaccatcagcagactggagcctgaagattttgcagtgtattactgtcagcagtatggtagctcacctccgtacacttttggccaggggaccaagccggagatcaaacgaactgtggctgcaccatctgtcttcatcttccc

SEQ ID NO: 18 - DNA, 386 bp, Homo sapiens; identical to SEQ ID NO: 17
  exon (1)..(386)

SEQ ID NO: 19 - DNA, 439 bp, Homo sapiens
  exon (1)..(439)
tgggttccaggttccactggtgaccaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacacactacaggtccaagtggtataatgattatgcagtatctgtgaaaagtcgaataaccatcaacccagacacatccaagaaccagttccccctgcagctgaactctgtgactcccgaggacacggctgtgtattactgtgcaagagacacagtgaggggaagtcagtgtgagcccagacacaaacctccctgcagggatgctcaggaccccagactccaccagctgaacctcgtcaccagtggaacctggaacccag

SEQ ID NO: 20 - DNA, 439 bp, Homo sapiens
  exon (1)..(439)
tgggttccaggttccactggtgaccaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagacacagtgaggggaagtcagtgtgagcccagacacaaacctccctgcagggatgctcaggaccccagactgcaccagctgcacctggtcaccagtggaacctggaacccag

SEQ ID NO: 21 - DNA, 439 bp, Homo sapiens
  exon (1)..(439)
gggttccaggttccactcggtgaccaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagacacagtgaggggaagtcagtgtgagcccagacacaaacctccctgcagggatgctcaggaccccagactccaccagctgcacctcgtcaccagtggaacctggaacccag

SEQ ID NO: 22 - DNA, 452 bp, Homo sapiens
  exon (1)..(452)
tgggttccaggttccactggtgacgaggttcagctggtggagtctgggggaggcgtggtccagcctgggaggtctctgagactctcctgtgcagcgtctggattcaaattcaatgactacggcatgcactgggtccgccaggctccagacaaggggctggagtgggtggcagccatttggtatgatggaactaacagatattatatagactccgtgaagggccgattcaccatctccagagacaattccaacagggcgctttatctccaaatgaacaacttgagaagcgaagacacggctgtctattattgtgtgaaagacgcgaatgttatgactggttattctgagtcgtggggccagggagtcctggtcatcgtctcctcagcctccaccaagggcccatcggtcttccccctggcaccctcctccaggagcacctctgggggcacagc

SEQ ID NO: 23 - DNA, 452 bp, Homo sapiens; identical to SEQ ID NO: 22
  exon (1)..(452)

SEQ ID NO: 24 - DNA, 452 bp, Homo sapiens
  exon (1)..(452)
tgggttccaggttccactggtgacgaggttcagctggtggagtctgggggaggcgtggtccagcctgggaggtctctgagactctcctgtgcagcgtctggattcaaattcaatgactacggcatgcactgggtccgccaggctccagacaaggggctggagtgggtggcagccatttggtatgatggaactaacagatattatatagactccgtgaagggccgattcaccatctccagagacaattccaacagggcgctttatctccaaatgaacaacttgagaagcgaagacacggctgtctattattgtgtgaaagacgcgaatgttatgactggttattctgagtcgtggggccagggagtcctggtcatcgtctcctcggcctccaccaagggcccatcggtcttccccctggcaccctcctccaggagcacctctgggggcacagc

SEQ ID NO: 25 - DNA, 452 bp, Homo sapiens; identical to SEQ ID NO: 24
  exon (1)..(452)

SEQ ID NO: 26 - DNA, 452 bp, Homo sapiens
  exon (1)..(452)
tgggttccaggttccactggtgacgaggttcagctggtggagtctgggggaggcgtggtccagcctgggaggtctctgagactctcctgtgcagcgtctggattcaaattcaatgactacggcatgcactgggtccgccaggctccagacaaggggctggagagggtggcagccatttggtatgatggaactaacagatattatatagactccgtgaagggccgattcaccatctccagagacaattccaacagggcgctttatctccaaatgaacaacttgagaagcgaagacacggctgtctattattgtgtgaaagacgcgaatgttatgactggttattctgagtcgtggggccagggagtcctggtcatcgtctcctcagcctccaccaagggcccatcggtcttccccctggcaccctcctccaagagcacctctgggggcacagc

SEQ ID NO: 27 - DNA, 452 bp, Homo sapiens
  exon (1)..(452)
tgggttccaggttccactggtgacgaggtgcagctggtgcagtctggaggaggcgtggtccagcctgggaggtctctgagactctcctgtgcagcgtctggattcaaattcaatgactacggcatgcactgggtccgccaggctccagacaaggggctggagtgggtggcagccatttggtatgatggaactaacagatattatatagactccgtgaagggccgattcaccatctccagagacaattccaacagggcgctttatctccaaatgaacaacttgagaagcgaagacacggctgtctattattgtgtgaaagacgcgaatgttatgactggttattctgagtcgtggggccagggagtcctggtcatcgtctcctcagcctccaccaagggcccatcggtcttccccctggcaccctcctccaggagcacctctgggggcacagc

(19) United States
(12) Patent Application Publication
(10) Pub. No.: US 2019/0056398 A1
(43) Pub. Date: Feb. 21, 2019

(54) IDENTIFICATION AND ISOLATION OF ANTIBODIES FROM WHITE BLOOD CELLS

(71) Applicant: SRI International, Menlo Park, CA (US)

(72) Inventors: Feng Wang-Johanning, Sunnyvale, CA (US); Gary L. Johanning, Sunnyvale, CA (US); Nathan Collins, San Mateo, CA (US); Xiaohe Liu, Palo Alto, CA (US); Zheng Ao, Fremont, CA (US); Lidia Sambucetti, Redwood City, CA (US)

(21) Appl. No.: 16/079,946

(22) PCT Filed: Feb. 24, 2017
(86) PCT No.: PCT/US2017/019478

§ 371 Date: Aug. 24, 2018

Related U.S. Application Data
(60) Provisional application No. 62/300,196, filed on Feb. 26, 2016.

(51) Int. Cl. G01N 33/569; C07K 16/18; C12N 5/0783; C07K 16/06; G01N 33/58

(52) U.S. Cl. CPC G01N 33/56972; C07K 16/18; C40B 20/08; C07K 16/065; G01N 33/582; C12N 5/0636

(57) ABSTRACT

Embodiments in accordance with the present disclosure include apparatuses, devices, and methods. For example, a method is directed to method exposing immobilized white blood cells from a blood sample to an antigen. And, scanning the immobilized white blood cells and, therefrom, identify and isolate white blood cells from among the immobilized white blood cells that produce an antibody in response to the exposure to the antigen.

### FEDERALLY-SPONSORED RESEARCH AND DEVELOPMENT

This invention was made with support by the Office of the Assistant Secretary of Defense for Health Affairs under Award W81XWH-12-1-0223. The U.S. Government has certain rights in this invention.

### INCORPORATION-BY-REFERENCE OF MATERIAL SUBMITTED ELECTRONICALLY

Incorporated by reference in its entirety is a computer-readable nucleotide/amino acid sequence listing submitted concurrently herewith, and identified as follows: One 57,922 Byte ASCII (Text) file named “SRII103PCT_Sequence” and created on Feb. 23, 2017.

### OVERVIEW

Antibodies are proteins that can be used by the immune system to detect, neutralize, and/or kill various target cells which may be harmful to the host organism, such as tumor cells and pathogens. The antibody can recognize and bind to a unique molecule of the target cell, called an antigen, via a binding region of the antibody. An antibody bound to the antigen can directly or indirectly (e.g., by triggering other parts of the immune system), detect, neutralize, and/or kill the target cell. For example, the binding may block a part of a microbe that is essential for the target cell to invade and survive. In other examples, the binding may impede biological processes causing the disease or may activate macrophages to destroy the target cell.

An antibody is generally a Y-shaped protein found in blood of humans and other vertebrates, and which belong to the immunoglobulin G (IgG) superfamily. There are five subclasses of antibodies, which include IgG, IgA, IgM, IgE, and IgD. IgG, the most abundant type of antibody, is found in all body fluids: it protects against bacterial and viral infections, and is commonly used as a cancer therapeutic antibody. Typically antibodies are made of various structural blocks and have two pairs of heavy chains and light chains. Each pair of a heavy chain and a light chain form a structure (e.g., like a lock) that fits a particle structure on an antigen, e.g., forms a binding region. While the general structures of different antibodies are similar, the binding region of the antibody is variable between the different antibodies and each of these variants can bind to different antigens. The heavy chains have one variable domain (V_{H}) followed by a constant domain C_{H}1, a hinge region and two more constant domains (C_{H}2 and C_{H}3). The light chains have one variable domain V_{L} and one constant domain C_{L}.

Antibodies that are produced from organism-specific B-cells can be used to treat various diseases and disorders for the organism (e.g., mammals, reptiles, birds, fish, and amphibians). As a specific example, antibodies produced from human B-cells, sometimes referred to as “fully human antibodies” or “humAb”, can be used to treat a human. Human B-cells can produce monoclonal antibodies, sometimes called “mAbs”, which mount immune responses and can minimize risks of cross reactivity with self-antigens. However, identifying fully human antibodies and other organism-specific antibodies that have the correct binding regions (e.g., correctly paired heavy and light chains) and have affinity for the target antigen can be laborious and unreliable.

The above issues as well as others have presented challenges to identifying and isolating human antibodies for a variety of applications.

### SUMMARY

The present invention is directed to overcoming the above-mentioned challenges and others related to the identifying and isolating antibodies from white blood cells (e.g., primary B-cells) as discussed above and in other implementations. The present invention is exemplified in a number of implementations and applications, some of which are summarized below as examples.

Various aspects of the present disclosure are directed to methods for screening and identifying antibodies secreted by white blood cells, such as B-cells. The identification can be directly from a whole blood sample from which red blood cells have been removed, sometimes referred to herein as “the white blood cell complement” or “whole white blood cell complement” for clarity purposes. The white blood cell complement of the blood sample is scanned to identify white blood cells. In specific embodiments, organism-specific antibodies (e.g., fully human antibodies) with correct binding regions, e.g., correctly paired heavy and light chains, for a target antigen can be identified from single white blood cells. Organism-specific antibodies, as used herein, refer to or include antibodies produced by B-cells of the organism. In specific embodiments, any organism which has blood (e.g., white blood cells) capable of producing antibodies as an immune (or other) response can be used to identify antigen-specific antibodies. The organism-specific antibodies are identified by scanning white blood cells from a blood sample (taken from the particular organism) and identifying antigen-specific white blood cells that produce therapeutic antibodies. In specific implementations, an optic scanner combined with a microengraving process can be used to simultaneously profile hundreds of thousands of white blood cells and assess the efficacy and cell functionality of the white blood cells. During the profiling, the white blood cells remain functional (e.g., are not killed), allowing for identified white blood cells to be selected by cell picking circuitry and equipment, and for further in vivo and in vitro processing and testing to be performed to further characterize the antibody, generate therapeutic antibodies, and/or for treatment of the organism.

A number of specific aspects are directed to methods of scanning a (whole) white blood cell complement of a blood sample to identify antigen-specific antibody producing white blood cells (e.g., B-cells). The method can include exposing immobilized white blood cells from the blood sample to an antigen. The white blood cell complement of the blood sample can be immobilized or fixed on a substrate and exposed to an antigen that is labeled. White blood cells (e.g., B-cells) that produce antigen-specific antibodies are identified by scanning the immobilized white blood cells. From the scan, white blood cells (among the immobilized) that produce an antibody in response to the antigen are identified as antigen-specific antibody producing white blood cells and are isolated. The antigen-specific antibody producing white blood cells are identified by locating respective white blood cells (B-cells) that have the labeled antigen bound on a surface of or near the white blood cell. The scan can be performed using an optic scanner that scans the entire white blood cell complement of a blood sample, such as at rates of between 1 million and 25 million cells per minute, although embodiments are not so limited. In response to identifying a white blood cell that produces an antibody, one or more fluorescent microscopes can be used to verify the antigen-specificity and, in some embodiments, to identify antigen-specific therapeutic antibodies. Moreover, the efficacy and cell function can be profiled, as further described herein.

Other specific embodiments are directed to a method for profiling a plurality of antibodies secreted by white blood cells for efficacy and cell function. The method includes use of a microengraving platform, wherein a substrate coated with an antigen is caused to contact a multiple-well array, such as a nanowell (or microwell) array, sometimes referred to as a “chip” or a “biochip”. The multiple-well array has a plurality of wells arranged in an array and each well contains an individual white blood cell. For example, the plurality of white blood cells can be deposited into the individual wells. Contacting the substrate to the multiple-well array can expose the plurality of white blood cells to the antigen. Each well of the multiple-well array can also include an individual target cell. A target cell, as used herein, includes or refers to cells that express the antigen. Example target cells include tumor cells and cells infected with bacteria or viruses (e.g., virus-infected cells and bacterial-infected cells). In specific embodiments, the white blood cells are co-cultured with the target cells within the multiple-well array (e.g., a nanowell array), and the white blood cells and target cells can be labeled. The substrate coated with the antigen is used to form an immuno-sandwich. For example, after incubating in contact with the multiple-well array, the substrate is treated with a labeled anti-organism (e.g., anti-human) detection antibody. Antibodies, that are produced responsive to the exposure to the antigen, can bind to the antigen coated on the substrate. The anti-organism detection antibody subsequently binds to the antibody, which can be detected via a scan of the substrate. The efficacy and cell function of the white blood cells can be assessed by scanning the substrate and the wells of the multiple-well array using an optic scanner and a fluorescent microscope. Further, white blood cells among the plurality that are antigen-specific and therapeutic (e.g., antigen-specific therapeutic antibodies) can be selected and isolated using cell picking circuitry.

Other specific embodiments are directed to an apparatus which includes the optic scanner, at least one fluorescent microscope, and cell picking circuitry. An example of an optic scanner can include a fiber optic bundle array, a laser, and imaging circuitry (e.g., camera). In specific aspects, the optic scanner can scan an entire white blood cell complement of a whole blood sample and generate a digital image of the location of white blood cells that produce an antibody responsive to a labeled antigen (e.g., identifying antigens bound to an antibody on a surface of or near white blood cells). The at least one fluorescent microscope of the apparatus can subsequently image the substrate to verify that the identified white blood cells have produced an antibody. The optic scanner and/or circuitry connected thereto can identify coordinates of the white blood cells that produce an antibody and provide the same to the fluorescent microscope for the subsequent imaging. In specific embodiments, the at least one fluorescent microscope includes two upright fluorescent microscopes. The cell picking circuitry can select white blood cells that are identified as positively producing an antibody.

The apparatus can include various additional circuitry such as processing circuitry for controlling the various instruments, memory circuit for storing data sets, and various computer-readable instructions for controlling the optic scanner, at least one fluorescent microscope, the cell picking circuitry and computer-executable instructions (e.g., software) for analyzing data obtained therefrom. In other specific embodiments, the apparatus additionally includes a microengraving platform, as previously described. The microengraving platform can be used to profile a plurality of white blood cells at the same time including analyzing the efficacy of the produced antibodies and the cell function of the white blood cells.

The above overview is not intended to describe each illustrated embodiment or every implementation of the present invention. The figures and detailed description that follow more particularly exemplify these embodiments.

### BRIEF DESCRIPTION OF THE DRAWINGS

Various example embodiments may be more completely understood in consideration of the following detailed description in connection with the accompanying drawings, in which:

FIG. 1 illustrates an example apparatus in accordance with various embodiments;

FIG. 2 illustrates an example process for identifying antigen-specific antibodies, in accordance with various embodiments;

FIG. 3 illustrates an example process for assessing cell efficacy, in accordance with various embodiments;

FIG. 4 illustrates an example process for identifying and assessing efficacy of antigen-specific antibodies, in accordance with various embodiments;

FIG. 5 illustrates an example of an optic scanner, in accordance with various embodiments;

FIG. 6 illustrates an example process for identifying and assessing efficacy of antigen-specific antibodies using a microengraving platform, in accordance with various embodiments;

FIG. 7 illustrates an example multiple-well array, in accordance with various embodiments;

FIGS. 8A-8C illustrate an example scan of a substrate of the microengraving platform as illustrated by FIG. 6, in accordance with various embodiments;

FIGS. 9A-9B illustrate example imaging of cells before and after isolation of an individual white blood cell, in accordance with various embodiments;

FIGS. 10A-10E illustrate example imaging of white blood cells, in accordance with various embodiments;

FIGS. 11A-11C illustrate example imaging of white blood cells by cell picking circuitry, in accordance with various embodiments;

FIG. 12A-12E illustrate example of amplification of B-cells, in accordance with various embodiments;

FIG. 13 illustrates example images of cells, in accordance with various embodiments;

FIGS. 14A-14C illustrate example images of a nanowell array, in accordance with various embodiments;

FIGS. 15A-15C illustrate example images of cells, in accordance with various experimental embodiments;

FIGS. 16A-16B illustrate example images of cells captured using an optic scanner and a fluorescent microscope, in accordance with various experimental embodiments;

FIGS. 17A-17E illustrate example images of a cell that is positive for CD19, human IgG, and a target antigen, in accordance with various embodiments;

FIGS. 18A-18C illustrate example images of a plurality of white blood cells which includes a B-cell that is positive for human IgG and a target antigen, in accordance with various experimental embodiments;

FIGS. 19A-19C illustrate example images of B-cells that are positive for human IgG and a target antigen, in accordance with various experimental embodiments;

FIGS. 20A-20C illustrate example images of B-cells that are positive for human IgG and a target antigen, in accordance with various experimental embodiments;

FIGS. 21A-21C illustrate a portion of a nanowell array as illustrated FIG. 7, in accordance with various experimental embodiments; and

FIGS. 22A-C illustrates white blood cells identified as killing cancer cells, in accordance with various embodiments.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of different types of devices, systems and arrangements used for identifying and isolating antibodies from white blood cells. White blood cells that produce antibodies can be identified by scanning white blood cells exposed to antigens and therefrom identifying white blood cells that produce antibodies. In certain implementations, aspects of the present disclosure have been shown to be beneficial when used in the context of scanning an entire white blood cell complement directly from an organism-specific blood sample, such as a human blood sample. In other implementations, nanoscreening is used to simultaneously profile hundreds of thousands of antibodies secreted by white blood cells and to assess the efficacy of the respective antibodies as well as interaction with target cells (for example, interaction with tumor cells as compared to normal cells). While the present invention is not necessarily limited to such applications, various aspects of the invention may be appreciated through a discussion of various examples using this context.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element.

Various embodiments in accordance with the present disclosure include apparatuses and methods for screening and/or isolating monoclonal antibodies (mAbs) produced by white blood cells that are organism-specific (e.g., fully human or other vertebrate such as a dog, cow, horse, fish or bird) and directly from blood samples. The white blood cells (e.g., B-cells) can be kept operational, and can be assessed to identify the phenotype of the white blood cells and to select detection antibodies, such as detection antibodies and/or therapeutic antibodies (TAbs). The white blood cells can be scanned using an optic scanner. The antibodies identified can be sequenced, cloned or otherwise amplified to generate antibodies (e.g., such as human IgG) that have correctly (matched) paired heavy and light chains for binding to the target antigen and can be used for diagnostic or treatment of the organism. In specific embodiments, the amplified antibody can be used to bind to the target antigen in vivo to treat a patient. For example, the identified antibodies are assessed to determine the cell function and binding efficacy. The selected and amplified antibodies can be antibodies that have illustrated an ability to bind to the antigen of the target cell and/or to neutralize (e.g., kill or prevent infection of) the target cell. Direct screening for antibodies against the antigen can be rapid (less than 2 weeks) and result in identification of mAbs and/or TAbs that are antigen-specific and that are organism-specific (e.g., fully human). In specific embodiments, the various methods for isolating antibodies can be used to identify a number of unique antibodies that are fully human, have correct binding sites specific to a particular antigen, decrease time to identify as compared to other techniques (e.g., screening libraries from immunized animals or synthetic phage or microbial libraries and/or isolating antibodies following white blood cell immortalization or cloning), and may not cause or reduce the risk of immune responses when humans are exposed to the identified antibodies.

The cloned antibodies can be used for treatment of the organism, such as for cancer treatment and/or treatment of viral and/or bacterial infections. For example, the antibodies identified can be cloned and the cloned antibody can used as a diagnostic agent, a sensor, and/or a therapeutic agent (e.g., a Tab). A number of TAbs used to treat cancer have been developed and used to treat and, in some cases, cure cancers. As a particular example, HER2-positive metastatic breast or ovarian cancer has been treated using Trasazumab™, which can increase patient outcomes. TAbs have a defined mechanism of action, have specificity and minimized off-target effects, and have predictable safety and toxicology profiles. While TAbs have many benefits, identifying fully human TAbs can be difficult as the antigen-specific antibodies within the whole human blood are rare (e.g., within 2000 or more white blood cells, one B-cells with antigen-specificity may be present). Further, in past instances, out of the many thousands of antibodies identified, few have the ability to bind to its molecular target, e.g., the antigen, with an affinity that is useful for neutralizing the target cells (e.g., killing the tumor cells). The efficacy of TAbs in treating tumor cells, for example, results from their ability to elicit potent tumor cytotoxicity either via direct induction of apoptosis in target cells or through effector-mediated functions like antibody dependent cell-mediated cytotoxicity (ADCC) and complement dependent cytotoxicity. For more general and specific information on the frequency of B-cells with antigen-specificity present in a blood sample, reference is made to “Frequencies of Cell Type in Human Peripheral Blood”, StemCell™ Technologies Canada Inc., https://www.stemcell.com/media/files/wallchart/WA10006-Frequencies_Cell_Types_Human_Peripheral_Blood.pdf, which is herein fully incorporated by reference.

Surprisingly, the various techniques described herein can be used to scan an entire white blood cell complement from a whole blood sample to identify B-cells with antigen-specificity. The scan can be rapid, e.g., up to 25 million cells per minute, by scanning using a fiber optic scanner, although embodiments are not so limited and can include other optic scanners, lower scan speeds (e.g., 10,000 cells per minute), and larger number of wavelengths. The rapid scan can increase screening throughput by 4,000 fold over other techniques. Responsive to the scan, identified B-cells with antigen specificity can be verified by imaging using fluorescent microscope. In a specific example, the methodology can be used to screen for cytotoxic antibodies against a tumor associated antigen called human endogenous retrovirus type K (HERV-K) envelope (env) protein. HERV-K can be expressed in breast and ovarian cancers. In an experimental embodiment, HERV-K-specific antibodies are identified and isolated in under two weeks and directly from human blood samples by simultaneously screening and profiling hundreds of thousands of antibodies secreted by B-cells from cancer patients. The profiling can include analysis of the efficacy of the B-cells, including the ability to bind to the HERV-K envelope protein and to kill cancer cells, all while the B-cells are located in the multiple-well array (e.g., a nanowell array). B-cells with the highest efficacy among the hundreds of thousand that are screened are isolated and their therapeutic effect is further verified, such as in vitro and/or in mouse models. Methods in accordance with the present disclosure allow for (i) direct screening of human antibodies (humAb), minimizing the possibility of anti-antibody immune responses, and (ii) discovery of pools of high-affinity antibodies by employing human samples known to contain titers of anti-antigen (e.g., anti-HERV-K env) antibody responses, coupled with high-throughput single-cell screening (100,000 to 400,000 B-cells) using nanowell screening, optic scanning, and fluorescent microscopy techniques.

In specific embodiments, a whole white blood cell complement from a blood sample is scanned. A white blood cell complement as used herein includes or refers to a blood sample (from a specific organism) with red blood cells removed, and which can contain white blood cells, platelets, and optionally plasma. The white blood cell complement can be immobilized or fixed to a substrate, such as one or more glass slide plates or in a (soft) matrix (e.g., agar or matrigel). A matrix can be used to maintain cell viability (e.g., the white blood cells are operational during and after the process). The substrate is treated with a labeled antigen, such as a fluorescently labeled antigen. White blood cells on the substrate that produce an antibody (and/or other therapeutic proteins) in response to exposure to the labeled antigen can bind to the antigen, and which can be identified from a scan of the substrate. For example, the antigen binds to the antibody, e.g., mAbs, either on the surface of or surrounding B-cells and fully differentiated plasma cells. In specific aspects, the white blood cell complement is activated prior to plating to encourage productions of antibodies. The white blood cell complement is scanned using the optic scanner. From the scan, white blood cells that produce antigen-specific antibodies are located and identified. The identified white blood cells are isolated as single cells using cell picking circuitry, such as commercially available AVISO CellCelector provided by Automated Lab Solutions GmbH. After isolating the white blood cells, the heavy and light chain sequences of the mAb are determined using PCR techniques, as further described herein. The antibody (e.g., mAb) can then be cloned into a production cell line, such as Chinese hamster ovary (CHO), to produce an antibody that can be further assessed for its binding affinity (e.g., antigen-specific binding affinity of the antibodies) and efficacy. This approach can allow for rapid identification of mAbs/TAbs directly from the white blood cell complement of a blood sample using the scan performed and for identifying rare cells in a large cell population. For example, the cloned antibodies can be used as a diagnostic agent, a sensor, and/or a therapeutic agent.

In other specific embodiments, white blood cells, which may be isolated as described above, are profiled using a multiple-well array, such as a nanowell array, and an apparatus as previously described. The nanowell array can be used to simultaneously screen and profile hundreds of thousands of antibodies secreted by white blood cells while also assessing the efficacy of antigen binding and target cell neutralization. The white blood cells are deposited into the nanowell array such that an individual white blood cell is located in each well of the array. The wells of the multiple-well array can be formed of fabricated polydimethyl siloxane (PDMS) and can maintain cell viability (e.g., via a cell culture media). The white blood cells are thereby operational when loaded into the wells. A combination of a microengraving process and an optic scan is performed to identify wells containing white blood cells that produce antigen-specific antibodies and to assess the efficacy of produced antibodies of the white blood cells. For example, the white blood cells can be assessed to identify the phenotype (e.g., therapeutic and/or diagnostic) and the ability to bind to the antigen.

Various other embodiments of the present disclosure are directed toward an apparatus used to perform the various methodologies described herein. The apparatus includes a (high speed) optic scanner, at least one fluorescent microscope or imaging system, and cell picking circuitry. As an example of an optic scanner is a fiber optic scanner, which includes a fiber optic bundle array, a laser, and imaging circuitry (e.g., camera), such as Fiber-optic Array Scanning Technology (FAST) as further described herein. The FAST fiber optic scanner scans a blood sample with the laser and collects a high resolution image of the sample using the fiber optic array. As previously described, the white blood cell complement of a whole blood sample is plated on a substrate (e.g., glass slide) and can be attached to a stage. The substrate is treated with a fluorescently labeled antigen and is scanned using the optic scanner. The optic scanner can scan the entire sample and generates a digital image of the location of white blood cells that produce an antibody responsive to the labeled antigen, which may be in sixty seconds in some experimental embodiments. The at least one fluorescent microscope of the apparatus can subsequently image the substrate to verify that the identified white blood cells have produced an antibody. The optic scanner and/or circuitry can identify coordinates of the white blood cells that produce an antibody and provide the same to the fluorescent microscope for the subsequent imaging. In specific embodiments, the fluorescent microscope includes two upright fluorescent microscopes. The cell picking circuitry can, responsive to the identification, select white blood cells that are identified as positively producing an antibody. And, the heavy and light chains of the antibody can be determined using single cell PCR techniques, as further described herein. The antibody can be cloned into a production cell line and further analyzed for antigen affinity and cell function. Although this disclosure describes scanning with the FAST system, embodiments are not so limited and one skilled in the art will recognize that other types of scanning can also serve the same purpose including those based on multispectral and/or hyperspectral imaging.

The apparatus can additional include various circuitry such as processing circuitry for controlling the various instruments, memory circuitry for storing data sets, and various computer-readable instructions for controlling the optic scanner, fluorescent microscope, cell picking circuitry and for analyzing data obtained therefrom. Optionally, in various specific-embodiments, the apparatus can include a microengraving platform. The microengraving platform includes a multiple-well array, an immuno-sandwich, and the at least one fluorescent microscope. As previously described, the multiple-well array and immuno-sandwich can be used to profile a plurality of white blood cells at the same time including assessing the efficacy of the produced antibodies and the cell function of the white blood cells.

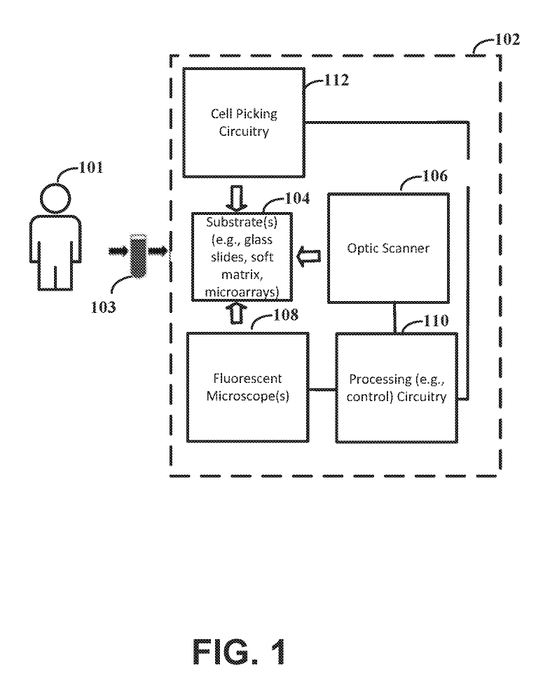

Turning now to the figures, FIG. 1 illustrates an example apparatus in accordance with various embodiments. The apparatus 102 can be used for scanning white blood cell complements of blood samples to identify antibodies that are organism-specific, such as fully human antibodies, and/or for profiling a plurality of antibodies secreted by white blood cells for efficacy.

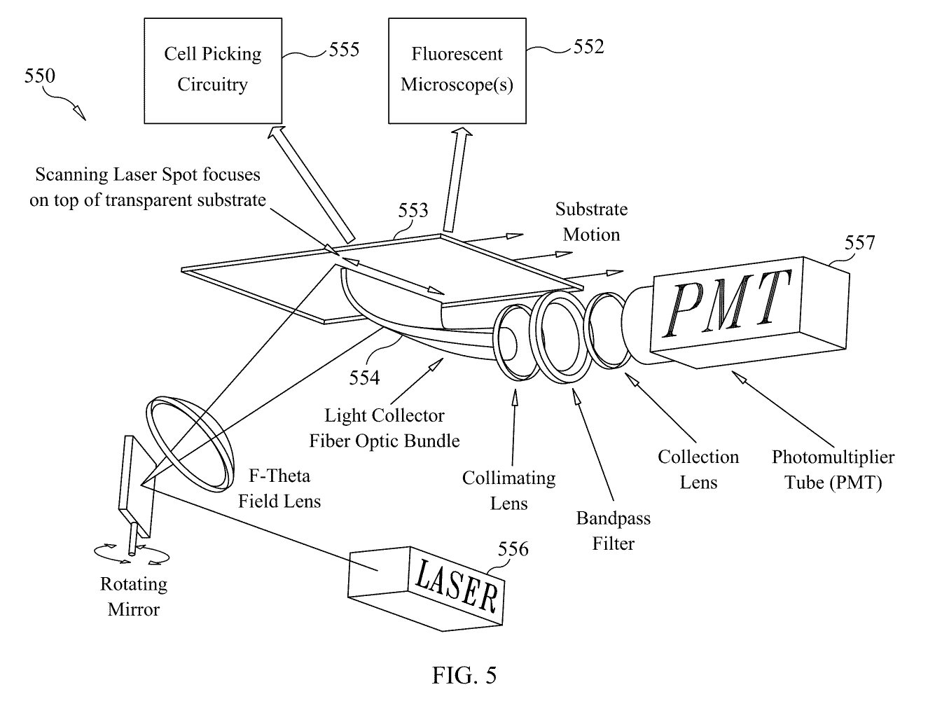

The apparatus 102 includes an optic scanner 106 combined with fluorescent microscope 108. The optic scanner 106 can include a platform termed FAST, as further illustrated and described in connection with FIG. 5. The optic scanner 106 can be used to directly identify novel diagnostic and/or therapeutic antibodies from blood samples immobilized on a substrate 104 (such as the blood sample 103 from a human 101 illustrated by FIG. 1) and to increase screening throughput by 4,000-fold over current antibody screening methods. An example scanning technology, the FAST technology is based on the concept of “Xeroxing” a blood sample with a scanning laser and collecting a high resolution capture image of the sample using a densely packed fiber optic array bundle. The FAST system can allow for rapid scanning of cells at speeds of between 1 million and 25 million cells per minute. For example, the optic scanner 106 can scan the substrate 104 containing or otherwise associated with a white blood cell complement of a blood sample. The white blood cells of the white blood cell complement are exposed to an antigen, either a labeled antigen treated directly on the substrate 104 and/or via formation of an immuno-sandwich.

The optic scanner 106 identifies white blood cells that produce an antibody responsive to exposure to the antigen and provides a location of the identified white blood cells to at least one fluorescent microscope 108. For example, using the optic scanner 106, antigens bound to an antibody at a surface of or near white blood cells are identified and used to identify the respective white blood cells. The optic scanner 106 can be in communication with fluorescent microscope 108, such as via processing circuitry 110 of the apparatus 102, to communicate coordinate locations of identified white blood cells that produce an antigen-specific antibody. In response to the coordinate locations, the fluorescent microscope 108 scans the identified white bloods cells to verify production of an antigen-specific antibody.

In some specific embodiments, the fluorescent microscope is used to further assess (e.g., profiles) the efficacy of the antigen-specific antibody and the cell function of the respective white blood cells. For example, the white blood cells can be deposited in a multiple-well array such that a single white blood cell is located in each well of the multiple-well array. A multiple-well array can include a microwell array and/or a nanowell array, among various other example arrays of wells. For ease of reference, the multiple-well array is here after referred to as the specific example of a nanowell array, however as may be appreciated by one of ordinary skill in the art, embodiments are not so limited.

The white blood cells can be from different sources (e.g., humans or other organism blood samples) and/or different white blood cells from the same source, as well as a combination thereof. The white blood cells in the nanowell array are co-cultured with individual target cells (e.g., tumor cells, virus-infected cells, bacterial-infected cells). For example, tumor cells from cancer patients can be used. Similarly to the white blood cells, a single tumor cell is deposited into each well. A glass substrate is coated with an antigen associated with the target cells (e.g., antigen expressed by the target cells) and used to form an immuno-sandwich by exposing the white blood cells located in the nanowell array to the antigen. The immuno-sandwich is used to detect antigen-specific antibodies secreted by the white blood cells. For example, the glass substrate is treated with a labeled anti-human detection antibody after incubating in contact with the nanowell array, and the optic scanner 106 scans the glass substrate to identify fluorescence indicative of the labeled anti-human antibody. If an antigen-specific antibody is produced by a white blood cell, the antibody binds to the antigen on the glass substrate and the anti-human detection antibody binds to the antibody. Subsequent detected fluorescence (associated with the labeled anti-human detection antibody) indicates that a respective white blood cell produced an antigen-specific antibody. Although the above example describes used of an anti-human detection antibody, embodiments are not limited to detection of human antibodies and can include detection of various other organism-specific antibodies, such as horse antibodies, dog antibodies, cat antibodies, fish antibodies, cattle antibodies, bird antibodies, among other organisms that have white blood cells which produce antibodies. As may be appreciated by one of ordinary skill in the art, the detection antibody used can be specific to the organism, such as an anti-horse detection antibody or an anti-dog detection antibody.

Optionally, in various embodiments, fluorescent microscope 108 scans the wells of the nanowell array to determine the phenotype of the white blood cells. As further described herein, the white blood cells can be tagged with a first fluorescence (e.g., blue) and the antigen of the target cells (located in the wells) can be tagged with a second fluorescence (e.g., orange). The fluorescent microscope 108 scans the wells containing the white blood cells that are co-cultured with the target cells and phenotypes the cells using the resulting fluorescent imaging. In specific embodiments, the ability of the white blood cells to kill the target cells can be identified based on the imaging. Data indicative of antigen-specificity captured via the optic scanner 106 and data indicative of the phenotype of the cells captured via the fluorescent microscope 108 can be mapped together. For example, as each spot on the glass slide corresponds to a well on the nanowell array, computer-readable algorithms are implemented by the processing circuitry 110 to map data captured by the fluorescent microscope 108 to data captured by the optic scanner 106.

In either embodiment, the cell picking circuitry 112 is used to isolate the identified white blood cells (individually) from the substrate 104. The cell picking circuitry 112 can include commercially available automated micromanipulators, such as the CellCelector available from Automated Lab Solutions GmbH.

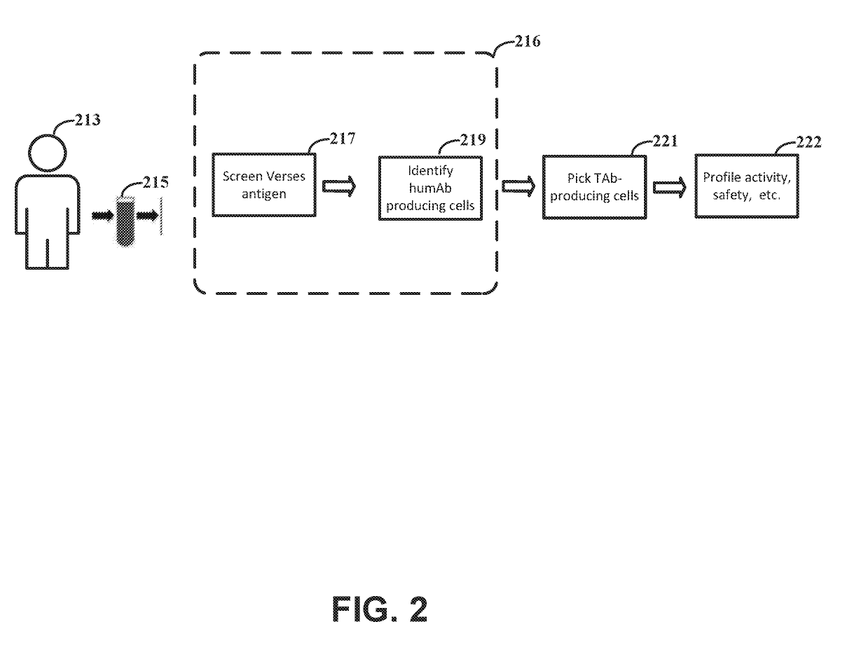

FIG. 2 illustrates an example process for identifying antigen-specific antibodies, in accordance with various embodiments. More specifically, FIG. 2 illustrates an example method for scanning a white blood cell complement from a whole blood sample to identify antigen-specific antibody producing white blood cells.

As illustrated by FIG. 2, a blood sample 215 is obtained from a human 213. The blood sample 215 can include a whole blood sample, in some embodiments. The whole blood sample is processed to remove red blood cells. In other embodiments, the blood sample has the red blood cells already removed. Although the embodiment illustrates the blood sample 215 being obtained directly from a human 213, embodiments are not so limited and the blood sample may be previously obtained and/or may be from other organisms and used to identify antibodies used to treat the particular organism (e.g., other vertebrates, such as horses, dogs, cats, cattle, fish, birds).

The white blood cells are immobilized, such as on a substrate, and attached to an apparatus 216. The apparatus 216 can include the apparatus previously illustrated and described by FIG. 1. In specific embodiments, the whole white blood cell complement for a blood sample is either fixed to one or more glass slide plates, or immobilized in a soft matrix such as agar or matrigel to maintain cell viability.

At 217, the immobilized white blood cells are screened verses an antigen. For example, the immobilized white blood cells can be exposed to an antigen. The exposure, in specific examples, includes treating the substrate with a (fluorescently) labeled antigen. White blood cells that produce antibodies responsive to the exposure can bind to the labeled antigen. For instance, the antigen binds to the produced human antibody (e.g., monoclonal antibodies (mAbs)) either on the surface of, or surrounding memory B-cells and fully differentiated plasma cells. The white blood cell complement may be activated before plating to ensure greatest production of antibodies.

At 219, the antibody producing white blood cells can be identified by scanning the immobilized white blood cells. From the scan, white blood cells are identified and isolated individually. The scan, in specific embodiments, can be by the optic scanner, such as a FAST scan of the substrate. The optic scanner identifies and locates the antigen specific human (e.g., mAb) producing B-cells. As previously described, at least one fluorescent microscope can be used to verify the antigen-specific antibody producing B-cells by further scanning the substrate. Data from the optic scanner and the fluorescent microscope is used to identify and isolate white blood cells that produce antibodies (e.g., human antibody or other organism-specific antibodies) responsive to the antigen exposure.

At 221, the antigen-specific antibody producing white blood cells are isolated as single cells by micromanipulation, such as a CellCelector micro aspiration technology. Using the isolated single white blood cells, the heavy and light chain sequences of the antibody are determined by single cell polymerase chain reaction (PCR) methods (as further described below). The antibody can then be cloned into a production cell line to produce antibody for binding affinity measurement and efficacy assessment, at 222. For example, the assessment can include assessing the antigen-specific binding affinity of the produced antibody, assessing the efficacy of the produced antibody, assessing the cell function of the white blood cell producing the antibody, and/or assessing the ability of the produced antibody to neutralize target cells associated with the antigen.

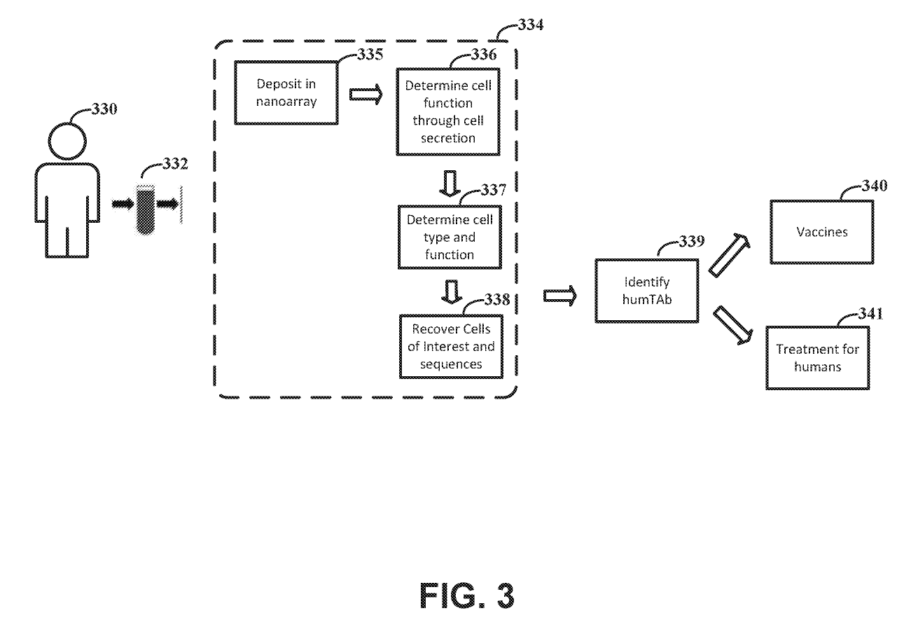

FIG. 3 illustrates an example process for assessing cell efficacy, in accordance with various embodiments. As previously described, an apparatus 334, such as the apparatus 102 illustrated by FIG. 1, can be used to simultaneously profile a plurality of white blood cells and produced antigen-specific antibodies. In specific examples, hundreds of thousands of B-cells can be simultaneously profiled.

As previously described, a blood sample 332 is obtained from an organism, such as a human 330 as illustrated although embodiments are not so limited. The blood sample 332 can include a whole blood sample, in some embodiments. The whole blood sample is processed to remove red blood cells. In other embodiments, the blood sample includes white blood cells isolated using the method illustrated and described by FIG. 2. Further, the white blood cells and/or the blood sample can be from a plurality of people.

The white blood cells from one or more blood samples are deposited into a nanowell array at 335. The nanowell array includes a plurality of wells arranged in an array, as further illustrated herein. Each well of the nanowell array can have an individual white blood cell deposited therein. Further, the wells can include a cell culture media that allows for the cells deposited in the wells to remain viable. A target cell is also deposited in each well of the nanowell array and the white blood cells are co-cultured with the target cells. In specific embodiments, the white blood cells can be labeled using one or more fluorescent labels and antigens express by the target cells can be labeled with a different fluorescent label. The fluorescent labels can be used for subsequent phenotyping and/or assessment of ability to kill the target cells. Optionally, in specific embodiments, white blood cells are incubated with antibodies against biomarkers C19/C20/C38. As a specific example, B-cells can be tagged for CD19+ with a first color and IgG+ with a second color (and the antigens of the target cells are labeled with a third color).

At 336, white blood cell function can be determined through cell secretion. For example, the white blood cells located in the nanowell array can be exposed to the antigen by causing a substrate coated with the antigen to contact the nanowell array. The substrate can be a poly-L-lysine glass slide having soluble antigens coated thereon and placed in contact with the nanowell array for a period of time (e.g., 2 hours). After incubating in contact with the nanowell array, the substrate can be treated with a labeled anti-human (or other anti-organism antibodies) Immunoglobulin G (IgG) antibody. The substrate is then scanned (e.g., using an optic scanner) to determine the cell function through secretion. For example, white blood cells that produce antigen-specific antibodies are identified from fluorescent emissions obtained via the scan. The scan by the optic scanner can be used to identify the coordinates on the glass slide that reveal discrete spots and that correspond to secretion of antibodies. As a specific example, if the white blood cell produces an antigen-specific antibody, the antibody binds to the antigen on the glass slide. The anti-human IgG antibody, which is labeled and washed over the glass slide, binds to the antibody and results in a fluorescent emission when scanned by the optic scanner. The coordinates on the glass slide are mapped to data obtained by the fluorescent microscope, as previously described.

At 337, the cell type and function is determined. For example, the fluorescent microscope scans the nanowell array, and the data from the scan is used to phenotype the white blood cells. The phenotype can include the type of cell (e.g., B-cell, T-cell) and the biological function of the cell. As an example, there are different functional states of white blood cells (e.g., B-cells and T-cells). In specific embodiments, particular interest is given to diagnostic cells that can be used to detect a disease or disorder and/or therapeutic cells that can neutralize or kill the target cells. Using the imagery (and based on the fluorescent labeling of the cells), white blood cells that produce antigen-specific antibodies and that neutralize (e.g., kill or prevent pathogen entry into) the target cell can be identified. Cytokines and cytotoxins such as perforin, granzymes, and granulysin secreted by cytotoxic T cells, which are white blood cells that kill cancer cells, can also be determined by coating the substrate with labeled antibodies that detect these secreted T cell factors. Further, as the plurality of white blood cells are simultaneously profiled, white blood cells that have the highest affinity and killing ability among the plurality of white blood cells can be selected for subsequent isolation.

At 338, the white cells of interest and sequences are recovered. As previously described, the particular white blood cells can be isolated individually using cell picking circuitry. At 339, the isolated white cells are used to identify an antibody and to further assess the antibody (as previously described in connection with FIG. 2). In specific embodiments, the identified antibodies can be diagnostic antibodies and/or therapeutic antibodies (TAbs). The antibodies are identified directly from the blood sample from a particular organism (e.g., vertebrate) and are thereby organism specific. In a specific example, as illustrated by FIG. 3, the TAbs are human TAbs (humTAbs). For example, the identified humTAb producing white blood cells are used to sequence the light and heavy chains of the humTAbs. The humTAbs, at 340 and 341, can be used to generate vaccines and other treatments for humans. In various experimental embodiments, the nanowell technology can be used to profile cell activity, to discover TAbs directly from blood of an organism, and to generate pharmaceutical drugs (e.g., vaccines or treatment).

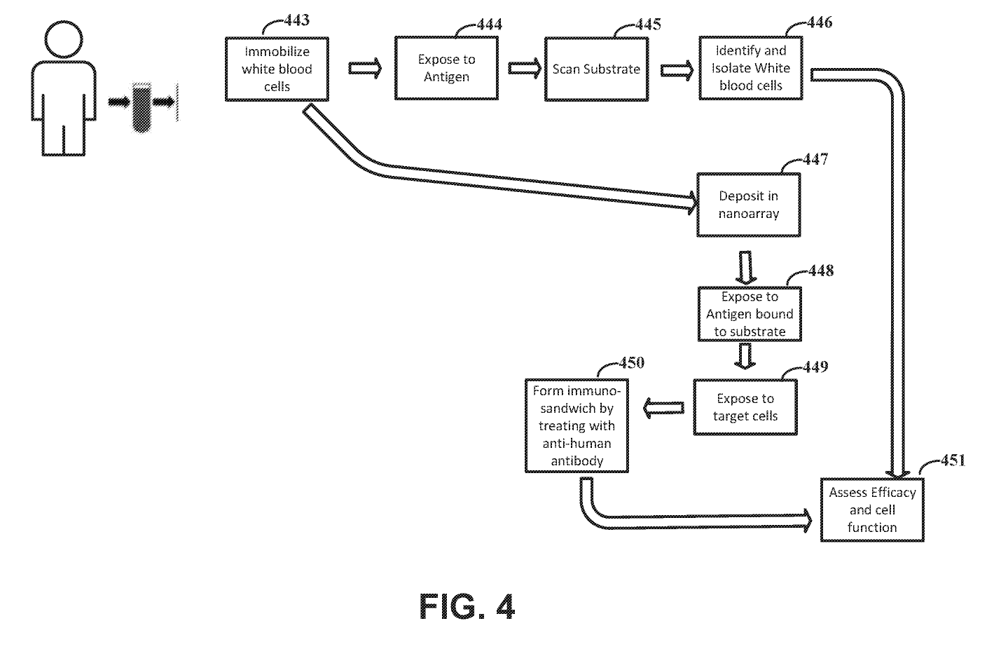

FIG. 4 illustrates an example process for identifying and assessing efficacy of antigen-specific antibodies, in accordance with various embodiments. In specific embodiments, the method illustrated by FIG. 2 can be used in combination with the method illustrated by FIG. 3 to identify antigen-specific antibody producing white blood cells and profile the antibodies secreted by the white blood cells. Further, the apparatus illustrated by FIG. 1 can be used, in various embodiments, to implement the processes illustrated by FIGS. 2, 3, and/or 4.

At 443, white blood cells from a blood sample are immobilized on a substrate. As previously described, the whole white blood cell complement from a whole blood sample can be analyzed by removing the red blood cells from the whole blood sample. The substrate can include a glass slide and/or a matrix (e.g., agar or matrigel). The immobilized white blood cells, at 444, are exposed to an antigen. The antigen can be labeled, such as with a fluorescent label.

At 445, the substrate is scanned to identify white blood cells from the immobilized white blood cells that produce an antibody (e.g., humAb). In specific embodiments, the substrate is scanned using an optic scanner (e.g., FAST system) that can scan the white blood cell complement of the whole blood sample at rates of up to twenty-five million cells per minute. White blood cells that produce an antibody can bind to the labeled antigen (e.g., bind on a surface or near) and emit fluorescence, responsive to the scan, due to the labeled antigen. In specific embodiments, the results can be verified using fluorescent microscopy. The optic scanner can identify the coordinates of the white blood cells that produce an antibody and provide the same to the fluorescent microscope. The fluorescent microscope can scan and image portions of the substrate to reduce or mitigate false positives.

At 446, the white blood cells that are identified (and, optionally, verified) as producing an antibody responsive to the antigen are isolated. For example, cell picking circuitry can isolate single white blood cells (e.g., isolate individually) using coordinates provided by the optic scanner.

The isolated white blood cells from the blood sample (and optionally, white blood cells from additional blood samples) can be used to identify antibodies with a threshold efficacy and cell killing ability. For example, at 447, a plurality of white blood cells, including the isolated white blood cells from step 446, are deposited into a nanowell array, although embodiments are not so limited. Each well of the nanowell array is populated with a single white blood cell. At 448, the plurality of white blood cells are exposed to an antigen. The antigen can be the same antigen as in step 444 above. In specific embodiments, exposing the white blood cells to the antigen can occur by causing a substrate coated with the antigen (e.g., an additional substrate) to contact the nanowell array. The specific antigen used is disease-specific. The plurality of white blood cells in the nanowell array, at 449, are exposed to target cells by causing each well of the nanowell array to include an individual target cell. The target cells and white blood cells in the nanowell array can be co-cultured to identify white blood cells (e.g., B-cells) capable of killing the target cells. In more-specific and related embodiments, the nanowell array is incubated with antibodies against CD19/CD20/CD38. The white blood cells and the target cells, located in the wells of the nanowell array, can be labeled for subsequent imaging. For example, the white blood cells can be tagged using a first fluorescence for CD19+ (e.g., first color) and a second (different) fluorescence for IgG+ (e.g., a second color). The target cells are tagged with the antigen (e.g., a third color). If antigen-specific antibodies are secreted by the white blood cells in the wells, then the antibodies bind to the antigen-coated glass cover slip, which is positioned over the microengraving plate and is in contact with the cell culture media in each well.

The substrate coated with the antigen can be placed in contact with the nanowell array for a period of time (e.g., 2 hours) and allowed to incubate. At 450, after incubation, the substrate is treated with a labeled anti-human (or other anti-organism) antibody. For example, the substrate can be washed and tagged with fluorescent anti-human IgG antibody. The anti-human IgG antibody can bind to antibodies present on the substrate and which are bound to the antigen that is coated on the substrate (e.g., on a surface of or near the white blood cells).

At 451, an efficacy and cell function of the plurality of white blood cells can be assessed. For example, the substrate, which is used to form an immuno-sandwich, is scanned using the optic scanner and the wells of the nanowell array are scanned using a fluorescent microscope (e.g., assess the antigen-specificity and phenotype). The scan of the substrate via the optic scanner can be used to identify antigen-specificity of the white blood cells. As previously described, each spot (e.g., fluorescent hit) of the substrate corresponds to antibody secretion from a white blood cell located in a single well of the nanowell array. The scan of the wells via the fluorescent microscope can be used to phenotype the white blood cells. In specific embodiments, the cell function of the white blood cell can be identified from the scan, including assessing the ability of the secreted antibody of the white blood cell to neutralize (e.g., kill) the target cells associated with the antigen. Cell picking circuitry can then be used to isolate single white blood cells from the plurality that produce antigen-specific TAbs (e.g., humTAbs). In specific embodiments, the isolated white blood cells are further processed and analyzed (as previously described in connection with FIG. 2). For example, the white blood cells are amplified for heavy and light chains of the TAbs and cloned into vectors to express TAbs, as further described herein.

Although the embodiments illustrated by FIGS. 2-4 describe identification of antigen-specific antibody producing white blood cells from a human, embodiments are not so limited. For example, in various embodiments, antigen-specific antibody producing white blood cells can be identified and used for treatment of other organisms, such as various vertebrates including dogs, cats, horses, livestock, birds, fish, etc.

FIG. 5 illustrates an example of an optic scanner, in accordance with various embodiments. The optic scanner illustrates is a fiber optic scanner. As illustrated the optic scanner 550 can be a portion of an apparatus, such as the apparatus illustrated in FIG. 1. The optic scanner 550 can be in communication with the fluorescent microscope 552 and cell picking circuitry 555 to form an apparatus that can identify antibody producing white blood cells from a blood sample and can profile the efficacy of a plurality of white blood cells.

The optic scanner 550 includes a light source (e.g., laser 556) to excite fluorescence located in a sample. The sample (e.g., blood sample) is immobilized or fixed to a substrate 553 and can be held in place by a stage of the optic scanner 550. In specific embodiments the light source is a laser 556, such as a 10 mW Argon laser that can excite fluorescence in labeled cells. The fluorescence can be collected in optics with a large (e.g., 50 mm) field-of-view. The field-of-view is enabled by an optic fiber bundle 554. The optic fiber bundle 554 can have asymmetric ends, in some embodiments, and the resolution of the optic scanner 550 can be determined by the spot size of the light source. The emissions from the fluorescent probe can be filtered through dichroic filters before detection at imaging circuitry 557, such as a photomultiplier. The substrate 553 can be moved orthogonally across the light scan path on the stage. The location of a fluorescently labeled cell is determined by the scan and the stage positions at the time of emission (and to an accuracy of ±70 um). For more specific and general information regarding an example FAST system, reference is made to Hsieh H B, Marrinucci D, Bethel K, et al., “High speed detection of circulating tumor cells”, Biosensors and Bioelectronics, 2006; 21: 1893-1899, and Krivacic R T, Ladanyi A, Curry D N, et al., “A rare-cell detector for cancer”, Proc Natl Acad Sci USA. 2004; 101: 10501-10504, each of which are fully incorporated herein by reference.

The optic scanner 550 illustrated can include FAST as implemented by SRI International, however embodiments are not so limited and other high speed scanning methods such as multispectral or hyperspectral imaging may be used. FAST was originally developed for the rapid detection of circulating tumor cells (CTCs), including enables high throughput scanning for fluorescently-labeled CTCs. Briefly, blood collected from patient and the red blood cells are lysed, and white cells are adhered and fixed to a pretreated glass slide and permeabilized for immunofluorescent labeling. After labeling, the slide is scanned, such as using laser-printing optics, an array of optical fibers that detects fluorescence emission from the cells.

CTCs are considered the seeds of residual disease and distant metastases, and their characterization could help to develop novel early detection markers, and may guide treatment options. FAST technology is a high-throughput, high-sensitivity scanner to scan all nucleated cells for an unbiased detection of CTCs on a planar substrate. The instrument enables rapid location of CTCs without the need for special enrichment, so its sensitivity is not degraded through, e.g., EpCAM targeted antibody enrichment. Because the sample preparation protocol does not distort cell morphology and CTCs are located on a planar surface, CTC imaging is of high fidelity, which leads to improved specificity. FAST also enables the simultaneous (multiplexed) analysis of multiple protein, cytogenetic, and molecular biomarkers at a single CTC level. Using FAST, it has been shown that the tumor marker human endogenous retrovirus type K (HERV-K) staining overlaps in many cases with staining of the serum tumor marker cytokeratin (CK), and suggest that HERV-K might be a CTC marker.

Once the tumor cells are identified and isolated, further investigation can examine the characteristics of single cells by immunohistochemistry and other analyses such as fluorescence in situ hybridization (FISH), polymerase chain reaction (PCR), and single nucleotide polymorphism (SNP) analysis.

In various embodiments, the apparatus including the optic scanner 550, the fluorescent microscope(s) 552 and the cell picking circuitry 555 can include additional circuitry. For example, the apparatus can include a server for storage of data sets, internal network connecting instrumentation control and database, and computer software for instrument control and data management (sometimes herein referred to as “processing circuitry” for ease of reference).

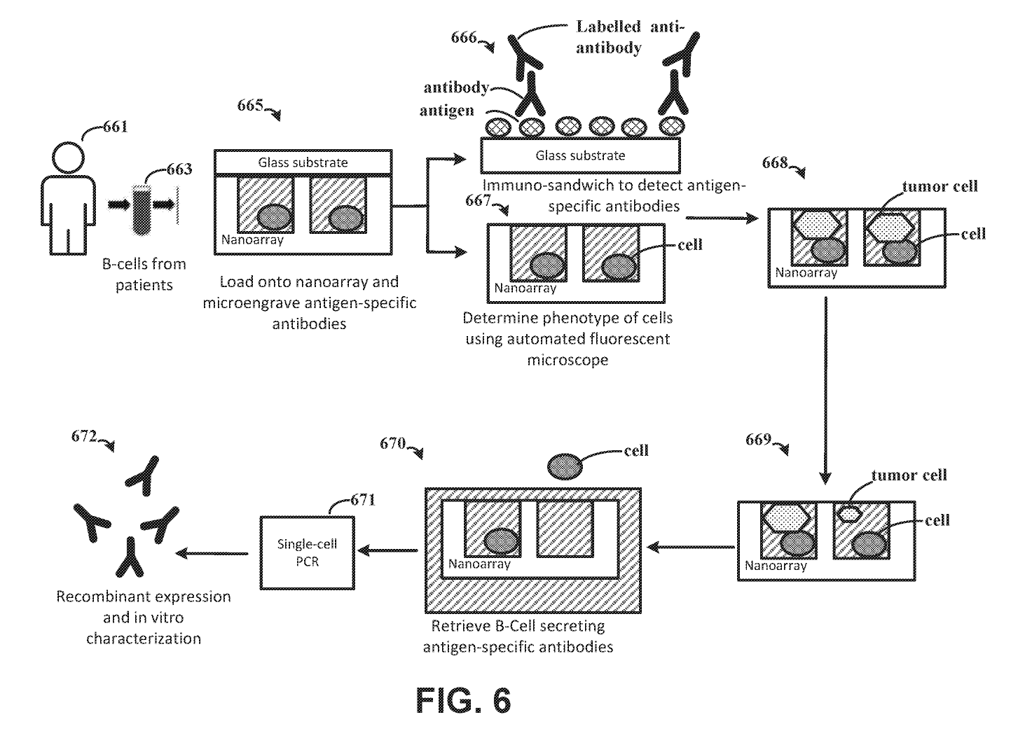

FIG. 6 illustrates an example process for identifying and assessing efficacy of antigen-specific antibodies using a microengraving platform, in accordance with various embodiments. As previously described, a microengraving platform can be used to profile the antigen-specificity and phenotype of a plurality of white blood cells at the same time. In some specific embodiments, human white blood cells (e.g., B-cells) can be screened for antigen-specificity and biological function using an optic scanner, fluorescent microscope(s), and the micro engraving platform. The microengraving platform can include a nanowell array and a glass substrate used to form an immuno-sandwich.

At 663, a blood sample from a human 661 can be obtained. The blood sample can be from a human that is known to have titers of antibodies against a target antigen in their sera. For example, blood samples can be drawn from breast cancer patients that have titers of antibodies against tumor antigens in their sera. The respective blood sample(s) and/or human can be selected by testing the blood for the antibodies. As further described below, white blood cells (e.g., B-cells) can be enriched from patient blood samples, and are stimulated using established protocols to promote antibody secretion, and determination of antigen positivity and production of antigen-specific antibodies in donor blood samples can be determined by RT-PCR, ELISA, and other immune assays.

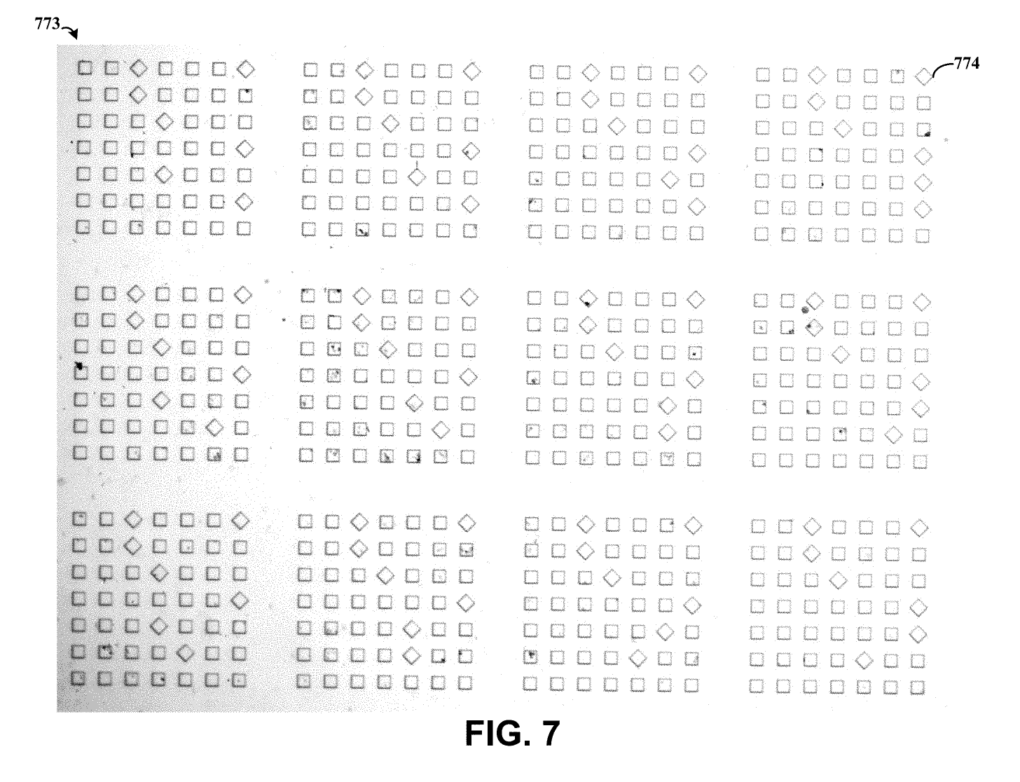

At 665, a plurality of white blood cells from the sample 663 (or a plurality of samples) are loaded into the nanowell array and antigen-specific antibodies are micro-engraved. The nanowell array, as further illustrated by FIG. 7, can include a plurality of wells, which can be fabricated by polydimethyl siloxane (PDMS). A single white blood cell is deposited into each well of the array. For example, 2×10⁵ B-cells can be loaded onto a nanowell array and the cells are allowed to settle via gravity. Soluable antigens are coated onto a substrate, such as a glass slide as illustrated. The substrate can include a poly-L-lysine glass slide, in specific embodiments. The substrate is placed in contact with the nanowell array for a period of time to allow for incubation, such as 2 hours. Further, as further illustrated at 668, the white blood cells in the wells of the nanowell array are co-cultured with individual target cells (e.g., tumor cells from a cancer patient) to identify white blood cells that are capable of killing the target cells. And, the nanowell array is incubated with antibodies against CD19/CD20/CD38.

Post incubation, at 666, the substrate is washed and tagged with a labeled anti-human antibody, such as fluorescently labeled anti-human IgG antibody, to form an immuno-sandwich and the substrate is scanned to identify fluorescent emissions that corresponds to secretion of antibodies by white blood cells. As illustrated, the immuno-sandwich can be formed by secreted antibodies binding to the antigen coated on the substrate and the labeled anti-human antibody binding to the antibody. In specific embodiments, the substrate can be scanned using an optic scanner (e.g., FAST system) to reveal discrete spots that correspond to secretion of antigen-specific antibodies by single B-cells. At 667, the phenotype of each of the white blood cells is determined using a fluorescent microscope. As each discrete spot on the substrate (e.g., glass slide) corresponds to antibody secretion from a single well on the array, algorithms are implemented via processing circuit to match the data obtained by the fluorescent microscope(s) to the data obtained by the fiber optic scanner to determine the antigen-specificity and phenotype of each of white blood cells on the nanowell array. As previously described, at 668, the white blood cells in the wells of the nanowell array are co-cultured with individual target cells (e.g., tumor cells from a cancer patient), which is used to identify white blood cells that are capable of killing the target cells, at 669 (e.g., illustrated by the tumor cell reducing in size between 668 and 669).

At 670, antigen-specific white blood cells are retrieved using cell picking circuitry (e.g., CellCelector). At 671, the variable regions of the isolated white blood cells are amplified using standard single-cell real time (RT)-PCR. And, at 672, recombinant expression and in vitro characterization are used to identify diagnostic and/or therapeutic antibodies with the requisite clinical properties.

FIG. 7 illustrates an example nanowell array, in accordance with various embodiments. As illustrated and previously described, the nanowell array 773 includes a plurality of wells arranged in an array (e.g., as illustrated by single well 774). The plurality of wells can be arranged on and/or attached to a solid surface. The nanowell array 773 (e.g., the wells) can be formed on polydimethyl siloxane (PDMS) using well known techniques. For more general information on nanowell array and specific information on forming nanowell array, reference is made to Varadarajan N, Julg B, Yamanaka Y J, et al, “A high-throughput single-cell analysis of human CD8+ T cell functions reveals discordance for cytokine secretion and cytolysis”, J Clin Invest, 2011; and Varadarajan N, Kwon D S, Law K M, et al., “Rapid, efficient functional characterization and recovery of HIV-specific human CD8+ T cells using microengraving”, Proc Natl Acad Sci USA. 2012; 109: 3885-3890, both of which are fully incorporated herein by reference.

In specific embodiments, white blood cells, such as from pre-screened patients (or other organisms being treated for a disease or condition) with titers of IgG responses, are loaded onto nanowell array 773 and the antigen specificity of the antibodies secreted by the white blood cells determined using substrates pre-coated with discovered antigens (e.g., a standard immuno-sandwich is used to identify these antigen-specific antibodies). The corresponding white blood cells are retrieved from the nanowells and the variable regions amplified using standard single-cell RT-PCR. Recombinant expression and in vitro characterization will be used to identify diagnostic and/or therapeutic antibodies with the requisite clinical properties.

Although the embodiment of FIG. 7 illustrates a nanowell array having wells of a particular shape and number, embodiments are not so limited. For example, the wells can include different shapes, orientation, and numbers than illustrated by FIG. 7. Example shapes include pyramids, three-sided pyramids, cones, prisms, rectangular prisms, among other shapes.

### More Specific/Experimental Embodiments

In specific experimental embodiments, HERV-K env, which is expressed by ovarian and breast tumor cells, is used as an antigen to identify antibodies. Using the above-described techniques, fully humAbs against HERV-K env can be identified in less than two weeks by nanowell screening to simultaneously profile hundreds of thousands of B-cells from cancer patients. Cancer patients having titers of anti-HERV-K antibodies are determined by using ELISA to detect the proteins and/or their antibodies in the blood sera of breast cancer patients. Patients with the highest-titers or titers above a threshold can be selected and their blood samples are used to identify fully human antibodies. Other proteomic assays can be used to confirm the findings by ELISA, such as an immunoblot. For example, an immunoblot can be used to detect anti-HERV-K antibodies in patient sera.

In specific experiment embodiments, to demonstrate that B-cells can be employed directly from blood samples of breast cancer patients (as a source of high-affinity antibodies), indirect ELISA with HERV-K-env recombinant fusion protein is performed. Peripheral blood mononuclear cells (PBMCs) from breast cancer patients can be polyclonally activated using irradiated 3T3-CD40L fibroblasts for a period of two weeks. This method can stimulate and expand CD40-B-cells to large numbers in a threshold purity (>90%) and induce secretion of their antibodies. Supernatants from the stimulated cultures can be used as the source of the antibodies in ELISA, which is used detect anti-HERV-K env specific responses from a number of different breast cancer patients, whose responses are stronger than those of ovarian cancer patients.

There are many advantages to using human B-cells to produce monoclonal antibodies: humans can mount powerful immune responses, and the antibodies are fully human, thus minimizing the risk of cross reactivity with self-antigens. Using the above-described apparatus, memory B-cells are scanned and antigen specific B-cells that generate diagnostic and/or therapeutic antibodies are identified by a microengraving process combined with FAST technology. Specifically, B-cells that produce antibodies that bind to HERV-K protein can be identified by incubating the HERV-K-producing B-cells with patient breast cancer cells in a microengraving plate, and incubating an HERV-K protein-coated cover slide that overlays the microengraving plate with goat anti-human IgG AF 555 to identify B-cells that bind to cancer stem cells isolated from the breast cancer cells.

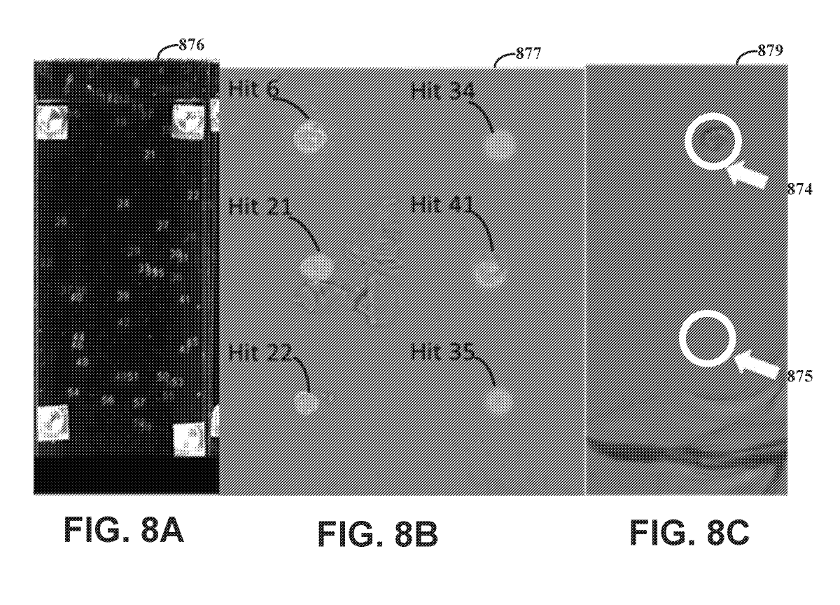

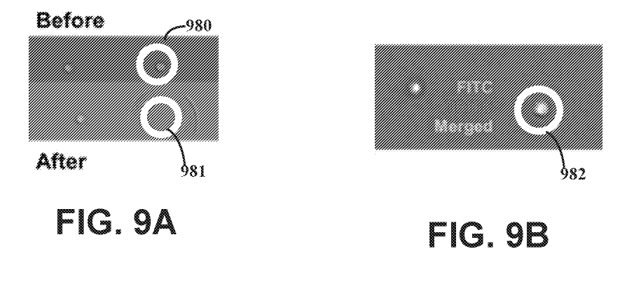

The arrays of nanowells in polydimethyl siloxane (PDMS) are fabricated, as previously described in connection with FIG. 7. PBMCs identified from pre-screened breast cancer patients with high titers of IgG antibodies against HERV-K Env proteins detected by ELISA or other immune assays, as described above, are stimulated using established protocols to promote antibody secretion from single B-cells. PBMCs are briefly stimulated ex vivo with B-cell stimulation cocktails for four days to facilitate the generation of antibody secreting cells. B-cells are loaded onto a nanowell array (one cell per well) and the cells allowed to settle via gravity. Soluble antigens are coated onto poly-L-lysine glass slides and placed in contact with the B-cell loaded nanowell array for two hours. Post-incubation, the glass slides are washed and tagged with fluorescent anti-human IgG antibody and read using an automated FAST system to reveal discrete spots that correspond to secretion of antigen-specific antibodies by single B-cells. Simultaneously, the nanowell array is incubated with antibodies against CD19/CD20/CD38 and the phenotype of every single cell on the chip is recorded using FAST. Since every single spot on the glass slide corresponds to antibody secretion from a single nanowell on the array, custom algorithms are implemented to match the data from the microscope to the data obtained from the FAST scanner to determine the antigen-specificity and phenotype of every B-cell on the nanowell array. One experiment, as further described herein, confirms that several hits scanned by FAST are B-cells that produced anti-HERV-K IgG antibodies. Antigen-specific B-cells can then be retrieved using an automated micromanipulator (CellCelector) for single-cell RT-PCR. Example results are shown by FIGS. 8A-8C and also by FIGS. 9A-9B.

FIGS. 8A-8C illustrate an example scan of a substrate of the microengraving platform as illustrated by FIG. 6, in accordance with various embodiments. Specifically, FIG. 8A illustrates an image 876 of the immuno-sandwich (as illustrated by FIG. 6) captured by an optic scanner, with the numbers indicating fluorescent hits. In specific embodiments, the antibody producing B cells specific to an antigen are captured and detected using anti-human IgG-Alexa555 by scanning with the optic scanner. FIG. 8B illustrates an image 877 of the immuno-sandwich captured by a fluorescent microscope used to confirm cell hits (e.g., as illustrated by cell hits 6, 21, 22, 34, 41, 34). FIG. 8C illustrates an image 879 of a single white blood cell captured by cell picking circuitry. The image 879 shows the single white cell 874 prior to isolation and the same location 875 of the substrate after the cell is isolated.

FIGS. 9A-9B illustrate example images of cells before and after isolation of an individual white blood cell, in accordance with various embodiments. FIG. 9A illustrates an image of a single B-cell 980 prior to isolation and the same location 981 after isolation by cell picking circuitry. FIG. 9B illustrates an example image of a HERV-K+ and IgG 982, which are respectively labeled using two different fluorescent labels (e.g., green and red). The single B cell clones with both the brightest green (HERV-K env+) and the brightest red (IgG+) fluorescence are detected and retrieved by the CellCelector for single cell RT-PCR. The heavy chain and light chain of RT-PCR products from a single B-cell is cloned and sequenced. Some sequence results are shown in the Sequence Listing named “SRII.103PCT Sequence”, as attached hereto.

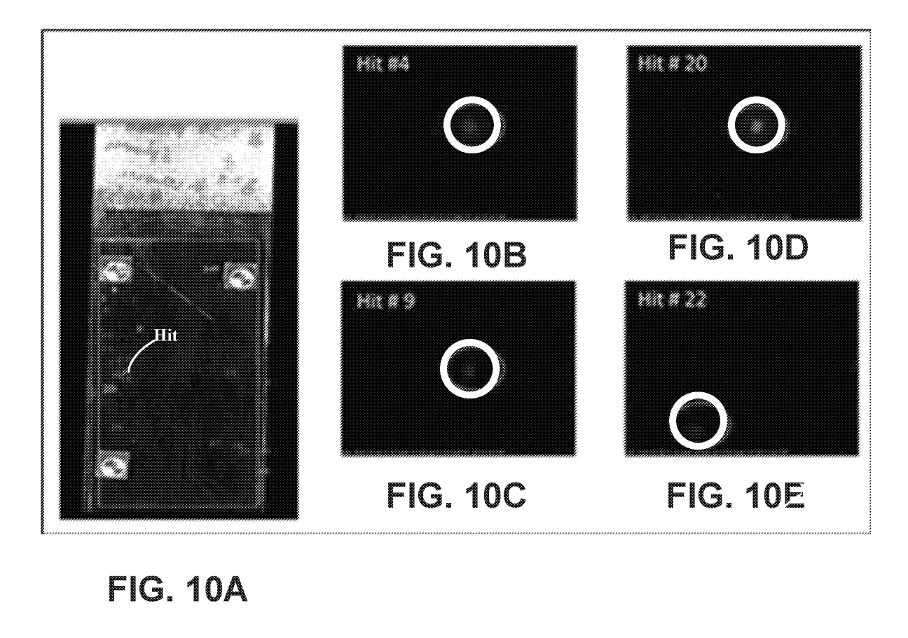

FIGS. 10A-10E illustrate example images of white blood cells, in accordance with various embodiments. In various experimental embodiments, a glass slide is coated with HERV-K protein and subsequently (e.g., the following day) the slide is washed and blocked with a blocking buffer (e.g., BSA). The blocking buffer can be removed and the glass slide is clamped to a microengraving plate that is plated with around 5000 HERV-K-stained B-cells from a particular breast cancer patient and co-incubated with around 2000 breast cancer cells from the same patient. The glass slide can be clamped onto the microengraving plate overnight. The following day, the glass slide is removed from the clamp and washed with 0.05 PBST and incubated with goat anti-human IgG AF 555. The glass slide is then washed and mounted with mounting media, covered with a cover slip and visualized on an optic scanner (e.g., FAST system). FIG. 10A illustrates an example image of the resulting glass slide as captured using an optic scanner. The white dots illustrate the fluorescent hits. The fluorescent hits can be confirmed using a fluorescent microscope. FIGS. 10B-10E illustrated images of the locations of the hits (e.g., hit #4, #9, #20, and #22) as captured by the fluorescent microscope (with the white circles highlighting the fluorescent hits).

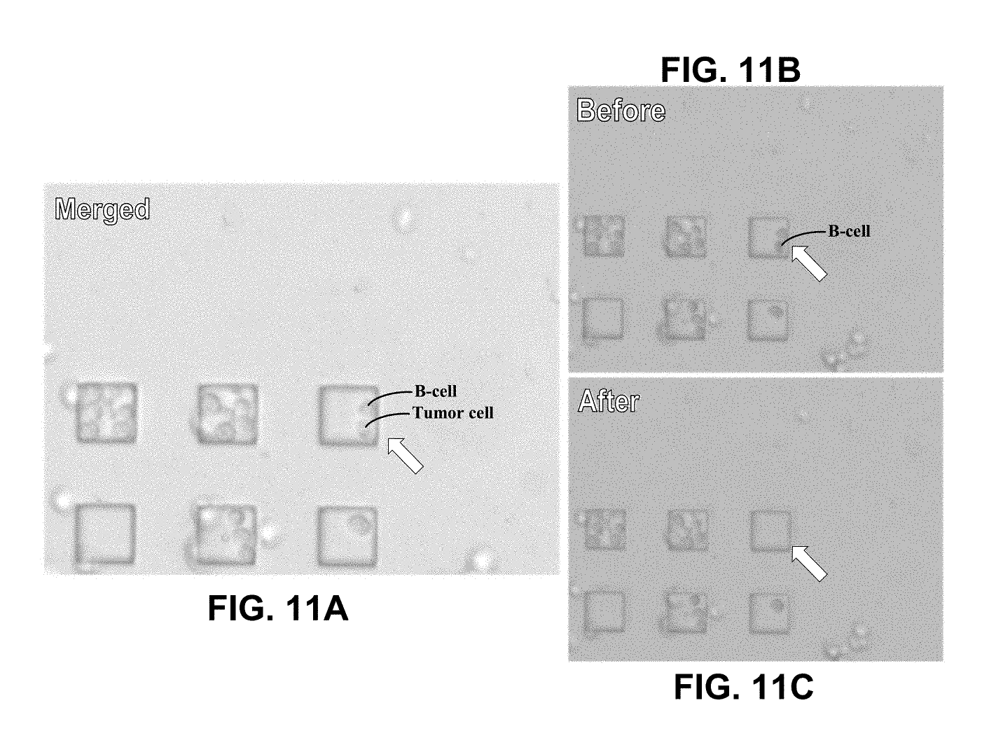

FIGS. 11A-11C illustrate example images of white blood cells by cell picking circuitry, in accordance with various embodiments. FIG. 11A illustrates a merged image of a portion of a nanowell array as imaged by cell picking circuitry. As highlighted, a cell is identified that includes a fluorescent B-cell (e.g., tagged in a red fluorescent). For example, the cell picking circuitry can select single B-cells for amplification of heavy and light antibody chains prior to cloning into vectors to express breast cancer therapeutic antibodies. FIG. 11B illustrates the well containing the B-cell as imaged prior to isolation and FIG. 11C illustrates the same well after the cell picking circuitry isolates the B-cell.

Once B-cells are identified and isolated, the B-cells can be amplified for further characterization. Amplification of the variable regions of the heavy and light chains of the candidate fully human antibodies can be performed using standard procedures that employ well-characterized oligonucleotides.

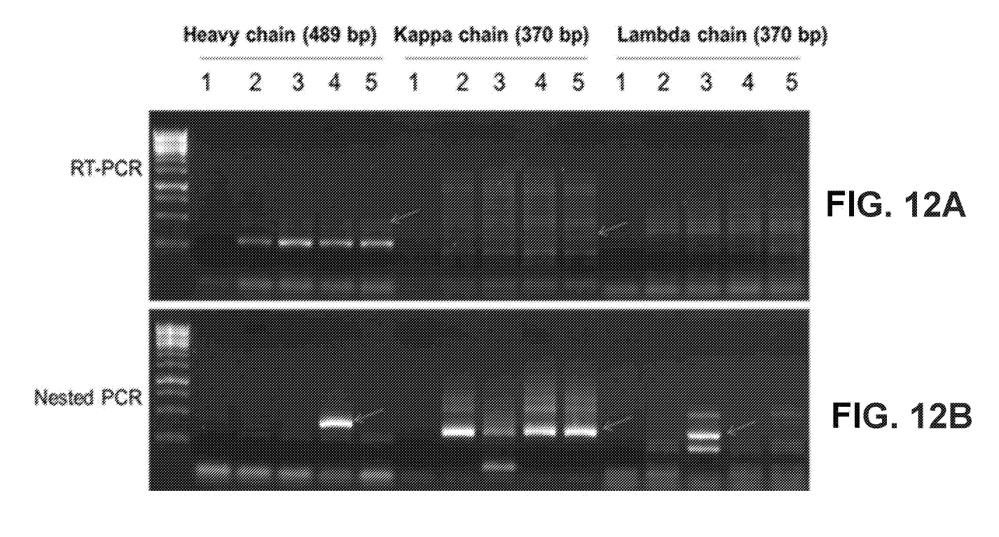

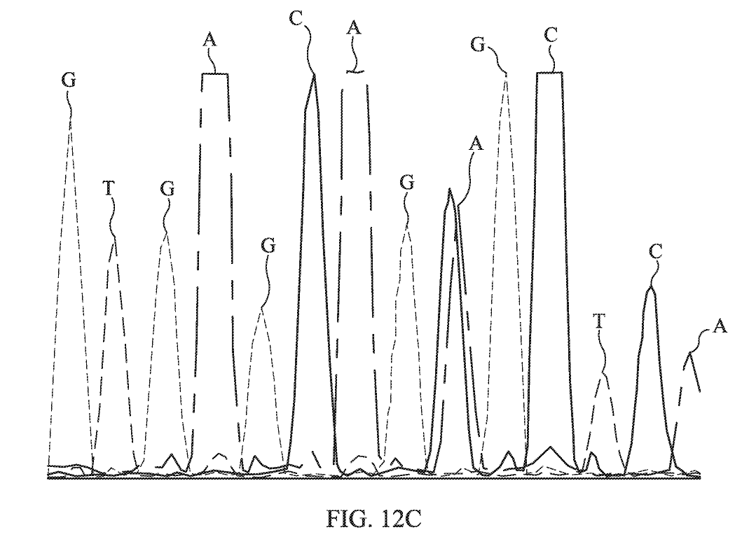

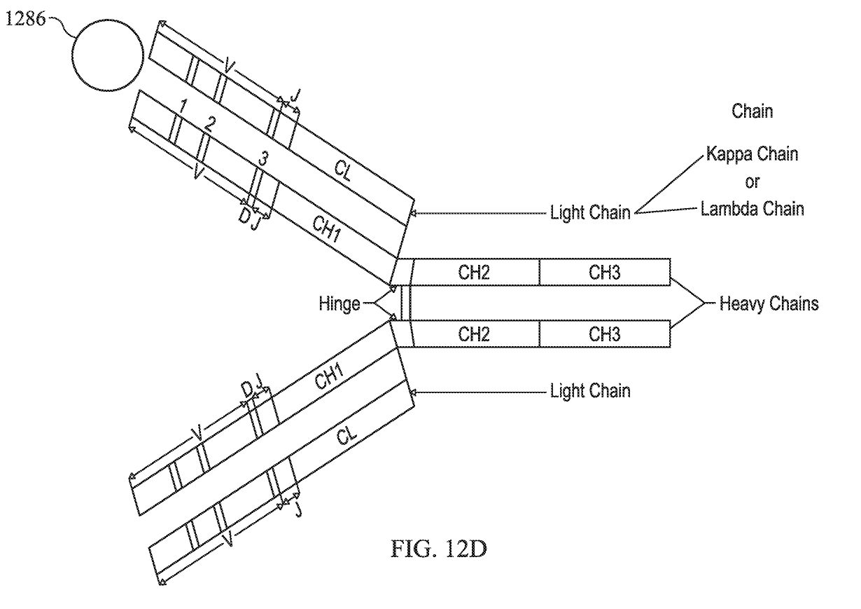

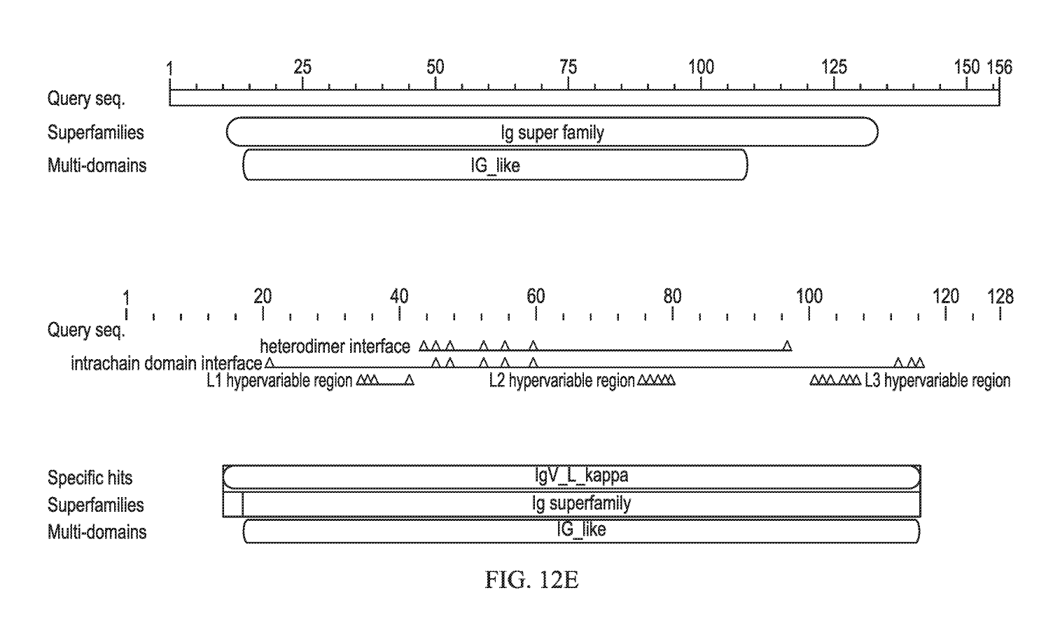

FIG. 12A-12E illustrate example of amplification of B-cells, in accordance with various embodiments. Both the variable heavy (VH) and variable light (VL) chains can be amplified by single-cell RT-PCR using standard procedures that employ well-characterized oligonucleotides. For more general and specific information on RT-PCR and Nested PCR, reference is made to Wang X, Stollar B D, “Human immunoglobulin variable region gene analysis by single cell RT-PCR. J Immunol Methods”, 2000; 244: 217-225; Liao H X, Levesque M C, Nagel A, et al., “High-throughput isolation of immunoglobulin genes from single human B cells and expression as monoclonal antibodies”, J Virol Methods, 2009; 158: 171-179; Sendra V G, Lie A, Romain G, Agarwal S K, Varadarajan N, “Detection and isolation of auto-reactive human antibodies from primary B cells”, Methods, 2013; 64: 153-159, each of which is fully incorporated herein by reference. Antigen-specific breast cancer patient B-cells are amplified down to single-cell resolution, as illustrated by FIGS. 12A and 12B. Single B-cell lysates are first reverse transcribed and amplified using a QIAGEN OneStep RT-PCR Kit, and 2.5 μl of the 1st PCR product is subjected to nested-PCR with TaKaRa Taq DNA Polymerase (commercially available from Clontech Laboratories Inc.), to amplify the variable regions of the IgG heavy chain or light chain separately, using degenerate primers. VH and VL amplicons are cloned into a pCR2.1-TOPO shuttle vector and subsequently re-cloned into isotype-specific heavy and light chain expression vectors to generate full-length human IgGs containing the VH and VL of interest, which are subsequently stably-transfected into CHO cells.

FIGS. 12A and 12B illustrate amplification of heavy, Kappa, and Lambda chains performed on five single B-cells. Specifically, FIG. 12A illustrates RT-PCR and FIG. 12B illustrates nested PCR.

The cloned PCR products, e.g., the cloned humAbs, can be further evaluated and/or processed. Examples of further evaluation and/or processing that can be performed include performing sequencing, and evaluating the efficacy of the antibodies. FIG. 12C illustrates an example experimental result of sequencing a PCR product. The sequences, such as illustrated by FIG. 12C, can be blasted to determine if there is a match to human IgG, as illustrated by FIG. 12D. FIG. 12D further illustrates the antigen 1286 binding to the antibody. FIG. 12E illustrates the results from the heavy chain (Ig superfamily) and the kappa chain (IgV L Kappa).

The specificity of the cloned humAb can be assessed using ELISA, cell ELISA, immunoblot, and immunocytochemical staining experiments performed on both tumor antigen-positive and tumor antigen-negative human breast cancer cells. For more general and specific information related to ELISA, cell ELISA, immunoblots, and immunocytochemical staining experiments, reference is made to Wang-Johanning F, Rycaj K, Plummer J B, et al, “Immunotherapeutic Potential of Anti-Human Endogenous Retrovirus-K Envelope Protein Antibodies in Targeting Breast Tumors”, J Natl Cancer Inst., 2012, which is fully incorporated herein by reference. In specific experiment embodiments, immunocytochemical staining experiments are imaged and scored. An ideal lead therapeutic candidate can show a statistically significant (P<0.05) increase in binding of tumor antigen-positive cells vs. tumor antigen-negative cells.

Employing the nanowell array based screening methodology can be used to stimulate and identify single memory B-cells from those patients that are capable of secreting antigen-specific antibodies. By using single-cell RT-PCR techniques, the paired variable regions corresponding to these antibodies are amplified and cloned. In addition, the cytolytic capability of B-cells can be determined by monitoring death of target cells (primary cells cultured from breast tumor tissues and from normal breast tissues as control cells), which is determined using SYTOX® (e.g., dead cells).

In addition to characterization by ELISA/Biacore (for affinity measurement), the antibody dependent cytotoxicity (ADCC) and complement dependent cytotoxicity (CDC)16 can be quantified using annexin V and caspase assays and antibody mediated induction of apoptosis can be quantified using caspase assays. For additional specific and general information related to annexin V and caspase assay, reference is made to Nechansky A, Szolar O H, Siegl P, et al, “Complement dependent cytotoxicity (CDC) activity of a humanized anti Lewis-Y antibody: FACS-based assay versus the ‘classical’ radioactive method—qualification, comparison and application of the FACS-based approach”, J Pharm Biomed Anal., 2009; 49: 1014-1020; and Dobson C L, Main S, Newton P, et al., “Human monomeric antibody fragments to TRAIL-R1 and TRAIL-R2 that display potent in vitro agonism”, MAbs, 2009; 1: 552-562, of which are herein both fully incorporated by reference.

Antitumor effects of human monoclonal antibodies can be tested in vitro and in vivo, as well as compared with murine mAbs side by side. Several breast cancer cells lines (MCF-7, MDA-MB-231, Hs578T, SKBR3, T47D, and primary BC cells) are used for in vitro assays. MCF-10A and MCF-10AT non-malignant primary breast cells can be used for controls. Multiple assays are carried out to determine the relationship between the expression of tumor antigen protein status after treatment with human or murine antibodies (or control antibodies) and cell growth, apoptosis and tumorigenic potential (soft agar assay) of breast cancer cell lines. Xenograft studies in NOD SCID mice or SRI's PDCellX models can also be performed to determine whether the breast cancer cells are less tumorigenic after treatment with antibodies directed against the tumor antigens, compared with the breast cancer cells treated with control antibodies, especially in reducing tumor size and metastasis to other organs.

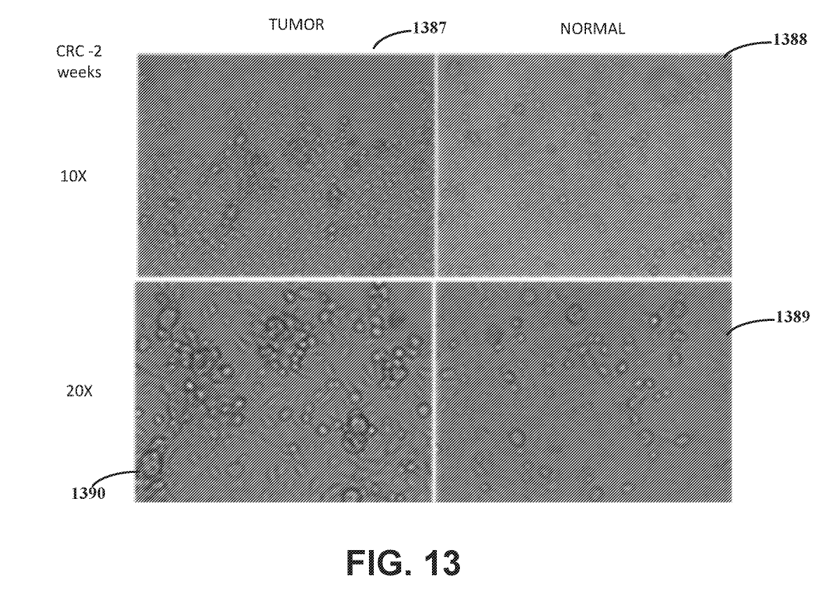

FIG. 13 illustrates example images of Conditional Reprogramming Cell culture (CRC) culture, in accordance with various embodiments. Normal primary breast cells from matched normal uninvolved breast tissues are illustrated by the images 1387, 1388, 1389, 1390 of FIG. 13A and other non-malignant breast cells including BRCA1+/2+ are cultured in lab using known CRC methods. For more general and specific information related to CRC methods, reference is made to Liu X, Ory V, Chapman S, et al., “ROCK inhibitor and feeder cells induce the conditional reprogramming of epithelial cells.”, Am J Pathol. 2012; 180: 599-607; and Ao Z, Parasido E, Rawal S, et al., “Thermoresponsive release of viable microfiltrated Circulating Tumor Cells (CTCs) for precision medicine applications”, Lab Chip, 2015; 15: 4277-428, both of which are fully incorporated herein by reference.

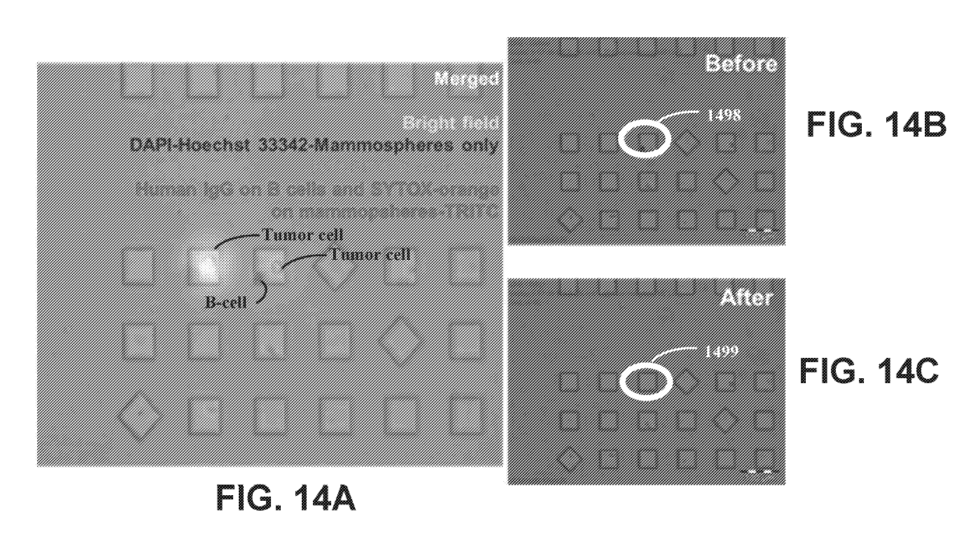

These CRC or mammosphere cells are used as targets for determining the efficacy of HERV-K specific B-cells. FIGS. 14A-14C illustrate example images of a nanowell array, in accordance with various embodiments.

FIG. 14A illustrates that the CRC or mammosphere cells are used as targets for determining the efficacy of HERV-K specific B-cells. Approximately 5,000 HERV-K stained B cells from a patient diagnosed with IDC are co-incubated with 2,000 HERV-K positive tumor mammospheres for 3-5 hours, and then picked using a CellCelector. Mammospheres are labeled with SYTOX orange, and HERV-K+ positive B cells are stained with Hoechst 33342.

FIGS. 14B and 14C illustrates the identification of B-cells that kill tumor mammosphere cells are picked up by a cell picking circuitry, and views before at 1498 (e.g., FIG. 14B) and after cell selection at 1499 (e.g., FIG. 14C) are shown. A single B-cell that killed the tumor cell is picked up by a CellCelector.

Some specific embodiments include an antibody identified directly from a human blood sample that specifically binds to HERV-K protein (e.g., HERV-K env protein). The antibody is thus fully human. The antibody can specifically bind to HERV-K protein (e.g., HERV-K env protein) for use in a method of treating cancer. For example, the antibody can specifically bind to HERV-K protein (e.g., HERV-K env protein) for use in a method of treating ovarian cancer, breast cancer, leukemia, lung cancer, melanoma, lymphoma, carcinoma, prostate cancer, among other types of cancer. The antibody which specifically binds to HERV-K protein (e.g., HERV-K env protein) can have the functional characteristic of killing target cells (e.g., cancer cells). In specific embodiments, the antibody specifically binds to HERV-K and can kill target cells while not killing normal cells (e.g., healthy cells). For example, the antibody can interact with tumor cells by killing the cells as compared to not interacting with normal cells and/or otherwise interacting with the normal cells that does not result in reduction of cell function (e.g., neutralization or killing). Further, as the antibody is fully human, the variable region can be used to mount immune responses and can minimize risks of cross reactivity with self-antigens. In specific embodiments, the antibody is detected and isolated from a sample from a single human (e.g., human blood sample). Further, the isolated antibody, including the variable region, can be isolated and sequenced from a single white blood cell. The variable region of the antibody has correct binding regions (e.g., correctly paired heavy and light chains) and an affinity for the target antigen due to the above-described methodology of detecting and isolating the antibody from a blood sample.

In further specific embodiments, the antibody can specifically bind to HERV-K protein (e.g., HERV-K env protein) for use in a method of treating cancer and which has the functional characteristic of killing target cells (and while not killing normal healthy cells), and can have variable regions as disclosed in the attached Sequence List. For example, the antibody can comprise a variable heavy (VH) region comprising SEQ ID NO: 19, 20, 21, 22, 23, 24, 25, 26, or 27 and a variable light (VL) region comprising SEQ ID NO: 1, 2, 3, 4, 5, 6, 7, 8, 9, 10, 11, 12, 13, 14, 15, 16, 17, or 18. In specific embodiments, the antibody can comprise VH region comprising SEQ. ID NO: 19, 20, or 21 and VL region comprising SEQ. ID NO: 13, 14, 15, 16, 17, or 18.

In accordance with such example experimental embodiments, HERV-K+, IgG+, and tumor-killing B-cells are screened and identified by the above-described apparatus, and picked up by cell picking circuitry for further characterization. More specifically, the molecular characterization of the anti-HERV-K protein antibodies (among other cancer TAbs, virus TAbs, and bacterial TAbs) can be identified and the effector functionality can be quantified in vitro using breast cancer cell lines. The antibodies that are isolated can be cloned into an Ig expression vector carrying the constant region of human gl, Ck, and Cl, sequenced and transiently expressed in CHO cells. In addition to standard characterization like ELISA/Biacore (for affinity measurement) the antibody dependent cytotoxicity (ADCC) and complement dependent cytotoxicity (CDC) can be quantified using annexin V and caspase assays, and antibody mediated induction of apoptosis can be quantified using caspase assays. Anti-tumor effects can also be determined in patient-derived xenograft models.

In accordance with various specific experiment embodiments, fully human antibodies against HERV-K Env protein, a model tumor antigen, are generated and their binding and antitumor effects are further determined in vitro and in vivo. Antibodies against tumor antigens and, more specifically, antibodies with specific targets for cancer and infectious diseases can be quickly identified (e.g., less than 2 weeks). In specific embodiments, sequences of the B-cells can be used to make fully human antibodies against the envelope protein of HERV-K. Further, methods can include developing purified fully human antibody against HERV-K envelope protein, and fully human anti-HERV-K Env antibody sequences. Such methodologies can be achieved using an apparatus that includes an optic scanner, fluorescent microscope(s) (e.g., two upright fluorescent microscopes), cell picking circuitry/equipment, and, optionally, a microengraving platform for identification of fully human antibodies. The antibodies identified and isolated can be used to generate a vaccine based on the fully human antibody polypeptides.

Although the above example describes identification of antibodies against HERV-K, embodiments are not so limited. For example, fully human antibodies to other proteins, such as Gag and Pol, as well as other HERVs and other target polypeptides, can be identified and isolated. The fully human antibodies can also be used to generate monoclonal cell lines that express human antibodies, diagnostic products based on the HERV-K Env polypeptide or its human antibodies, therapeutic treatment based on the human antibodies, and/or cures for the recurring illness. The apparatus, for example, can be used for producing TAbs that target other infectious disease-associated antigens that are capable of inducing an immune response. For example, TAbs are produced against influenza hemagglutinin antigen and antigens associated with Zika or Ebola. Individual B cells from infected patients are binned into nanowells on a plate (FIGS. 1 and 2), and antibody production against the antigen target is used as a guide for selecting and amplifying antibodies from B cells that show especially strong positive responses. The TAbs are tested in vitro using virus neutralization assays, and in animal models of the infectious disease for efficacy in blocking infection.

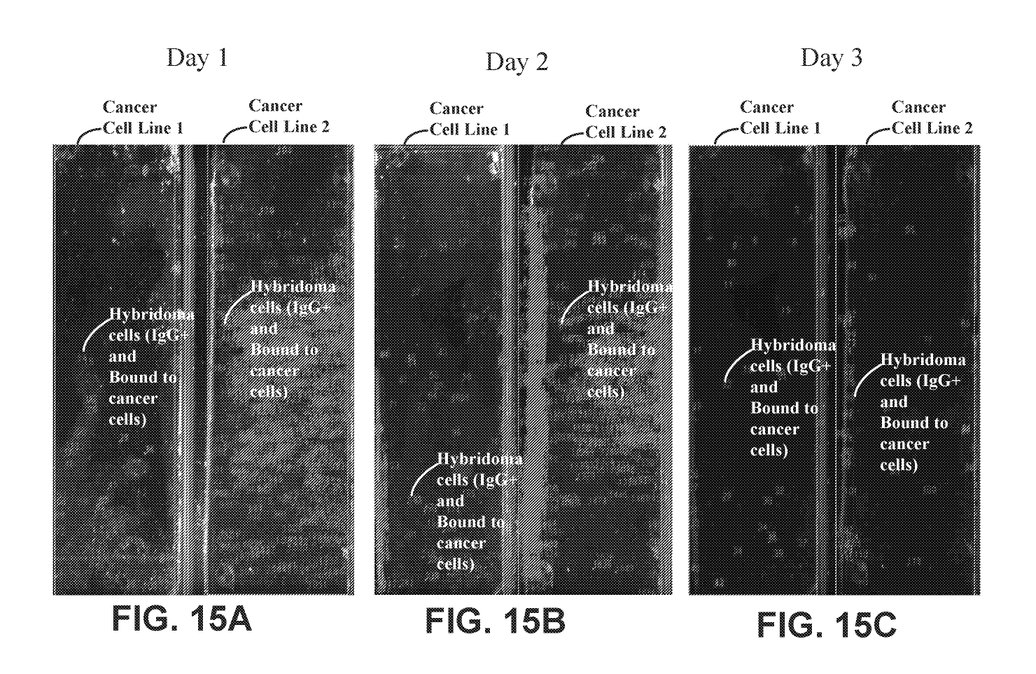

FIGS. 15A-15C illustrate example images of cells from experimental embodiments, in accordance with various embodiments. As illustrated, an example antibody that specifically binds to HERV-K (e.g., anti-HERV-K monoclonal antibody) is co-cultured with two types of cancer lines. In specific embodiments, the two types of cancer lines include triple negative breast cancer (TNBC) cell lines, such as Hs578T and MDA-MB-231, that are co-cultured with hybridoma cells (e.g., antibodies). The hybridoma cells can be co-cultured with the TNBC cell lines for one day, two, and three days as respectively illustrated by FIGS. 15A, 15B, and 15C. Cells (e.g., hybridoma cells) that are positive both human IgG (huIgG) and binding to cancer cells produce a wavelength when imaged and is identified as a hit (e.g., red or other fluorescent color). The hybridoma cells identified as hits are illustrated in each of FIG. 15A-15C by a representative hybridoma cell that is labeled. Although only one hybridoma cell is labeled, for clarity purposes, multiple hits are illustrated in each image (e.g., which are in red when viewed in color and grey-numbers and/or grey-scale when viewed in black and white). As illustrated by FIG. 15A, on day 1, the cancer cell line 2 has more hybridoma cell hits relative to the cancer cell line 1. On day 2, as illustrated by FIG. 15B, a reduced number of hybridoma cell hits is observed for both cancer cell lines 1 and 2 as compared to day 1 (FIG. 15A) and which indicates that the cancer cells are being reduced (e.g., killed by the hybridoma cells). As illustrated by FIG. 15C, a reduced number of hybridoma cell hits is observed for both cancer cell lines 1 and 2 as compared to day 1 (FIG. 15A) and day 2 (FIG. 15C). The second cancer cell line (MDA-MB-231 cells) in specific experimental embodiments results in a greater number of hybridoma cell hits (double positive for huIgG and bound to cancer cells) relative to the reduction in cancer cell line 1.

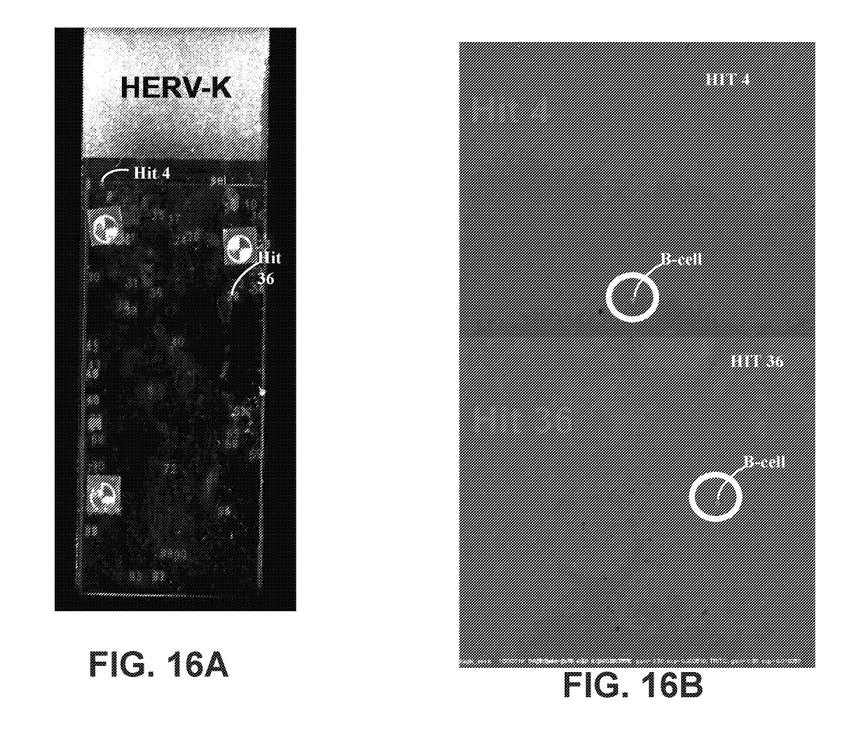

FIGS. 16A-16B illustrate example images of cells captured using an optic scanner and a fluorescent microscope, in accordance with various experimental embodiments. As illustrated by FIG. 16A, an optic scanner is used to detect cells which are antigen-specific antibody producing B-cells (e.g., positive for both human IgG (huIgG+) and HERV-K (e.g., HERV-K env+)). FIG. 16B illustrates imaging by a fluorescent microscope used to verify that the detected B-cells produce antigen-specific antibodies (e.g., cells that hit as positive for both huIgG and HERV-K).

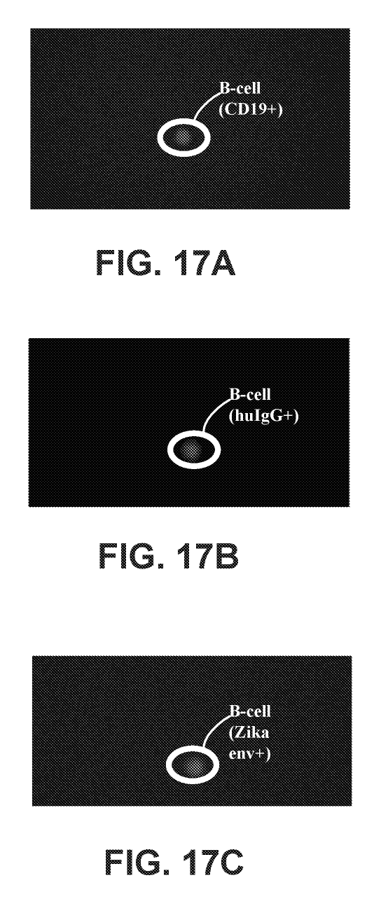

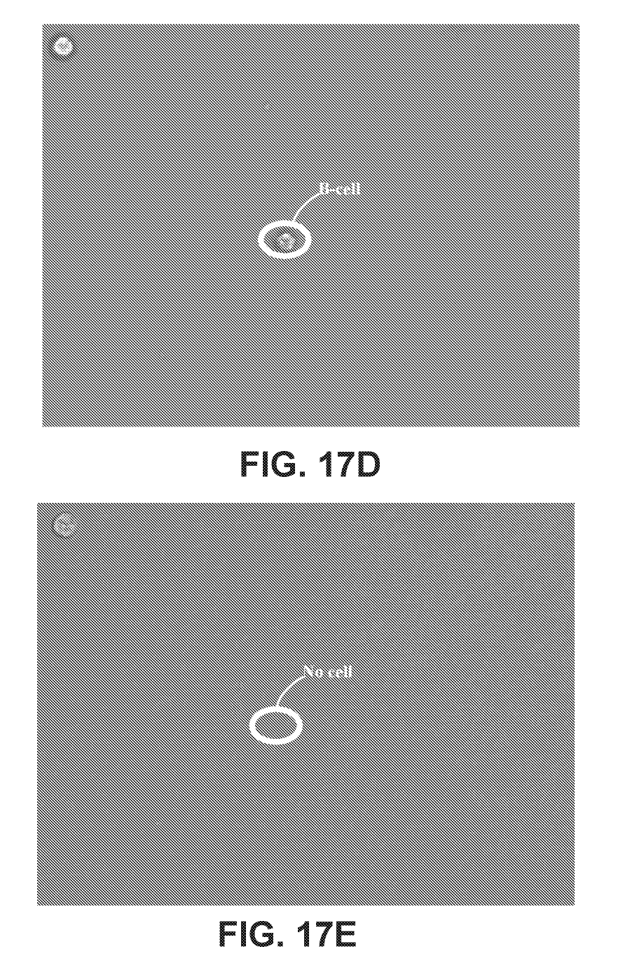

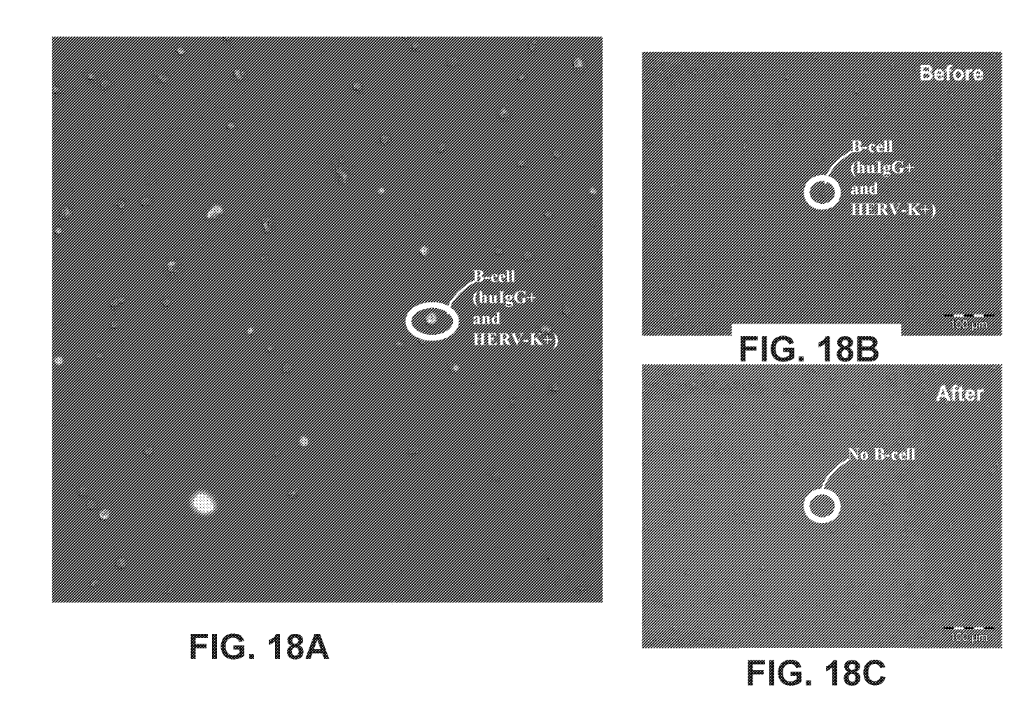

FIGS. 17A-17E illustrate example images of a cell that is positive for CD19, human IgG, and a target antigen (e.g., Zika env), in accordance with various embodiments. For example, the images illustrated by FIGS. 17A-17C illustrate the same B-cell which is identified as being positive for CD19, huIgG, and Zika env (each respectively resulting in a different fluorescent hit, such as blue, green, and red). FIG. 17D-17E illustrate an image of the B-cell (that was triple positive for CD19, huIgG, and Zika Env) as detected (FIG. 17D) and the substrate after the cell is isolated (FIG. 17E) using cell picking circuitry. FIGS. 18A-18C illustrate example images of a plurality of white blood cells which includes a B-cell that is positive for human IgG and a target antigen (e.g., HERV-K env), in accordance with various experimental embodiments. Specifically, FIG. 18A illustrates an example image of a plurality of white blood cells. The white blood cells are treated with a fluorescent tag (e.g., red) used to identify B-cells and a fluorescently-tagged antigen (e.g., green). White blood cells that are positive for huIgG and the antigen (e.g., produce a fluorescent hit (color) that includes both tags, such as a wavelength the results from combining the two tags) are detected. FIG. 18B-18C illustrate an image of a B-cell that is detected as being positive for huIgG, and HERV-K (FIG. 18B) and the substrate after the B-cell is isolated (FIG. 18C) using cell picking circuitry.

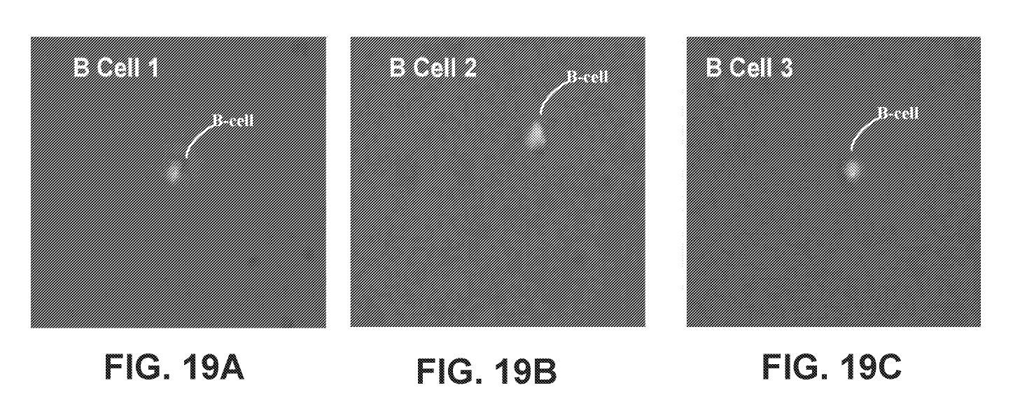

FIGS. 19A-19C illustrate example images of B-cells that are positive for human IgG and a target antigen (e.g., HERV-K env), in accordance with various experimental embodiments. Specifically, FIGS. 19A-19C illustrates three B-cells detected that are positive for human IgG and HERV-K that were confirmed using a fluorescent microscope (e.g., examples of DAPI, TRIC, and B-cells identified on a substrate).

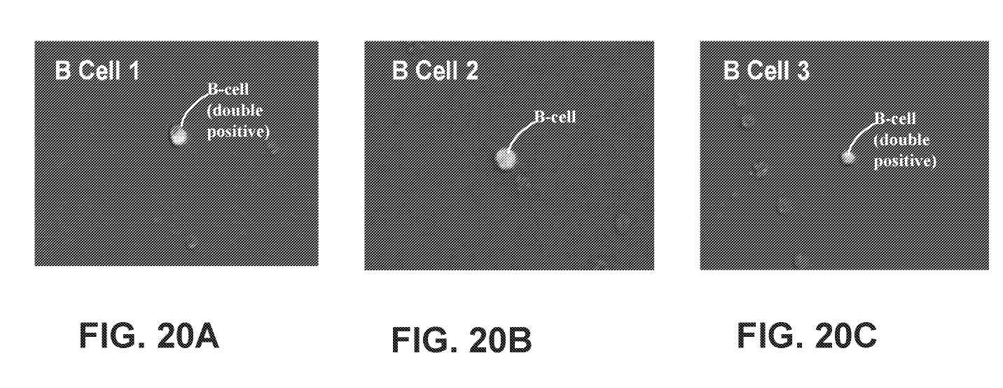

FIGS. 20A-20C illustrate example images of B-cells that are positive for human IgG and a target antigen (e.g., HERV-K env), in accordance with various experimental embodiments. Specifically, FIGS. 20A and 20C illustrate two B-cells detected that are positive for human IgG and HERV-K and this confirmed using a fluorescent microscope (e.g., examples of DAPI, TRIC, and B-cells identified on a). FIG. 20B illustrates a B-cell detected that is not double positive (e.g., only positive for huIgG).

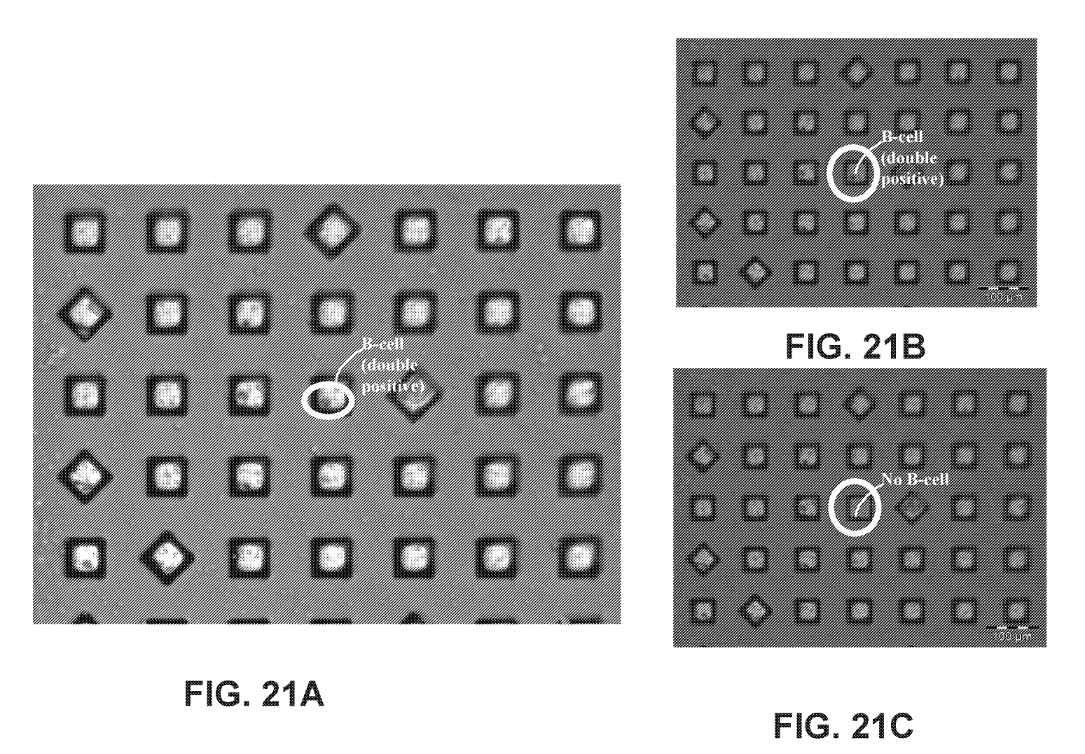

FIGS. 21A-21C illustrate a portion of a nanowell array (e.g., microengraving plate) as illustrated FIG. 7, in accordance with various experimental embodiments. More specifically, FIG. 7 illustrates examples of B-cells that are detected as being positive for IgG and a target antigen (e.g., positive for huIgG and HERV-K env) by a fiber-optic scanner and confirmed by a fluorescent microscope. FIG. 21A illustrates an example B-cell that is positive for huIgG and HERV-K as detected. FIG. 21B-21C illustrate an image of the B-cell (that is positive for huIgG and HERV-K) as detected (FIG. 21B) and the well after the B-cell is isolated (FIG. 21C) using cell picking circuitry.

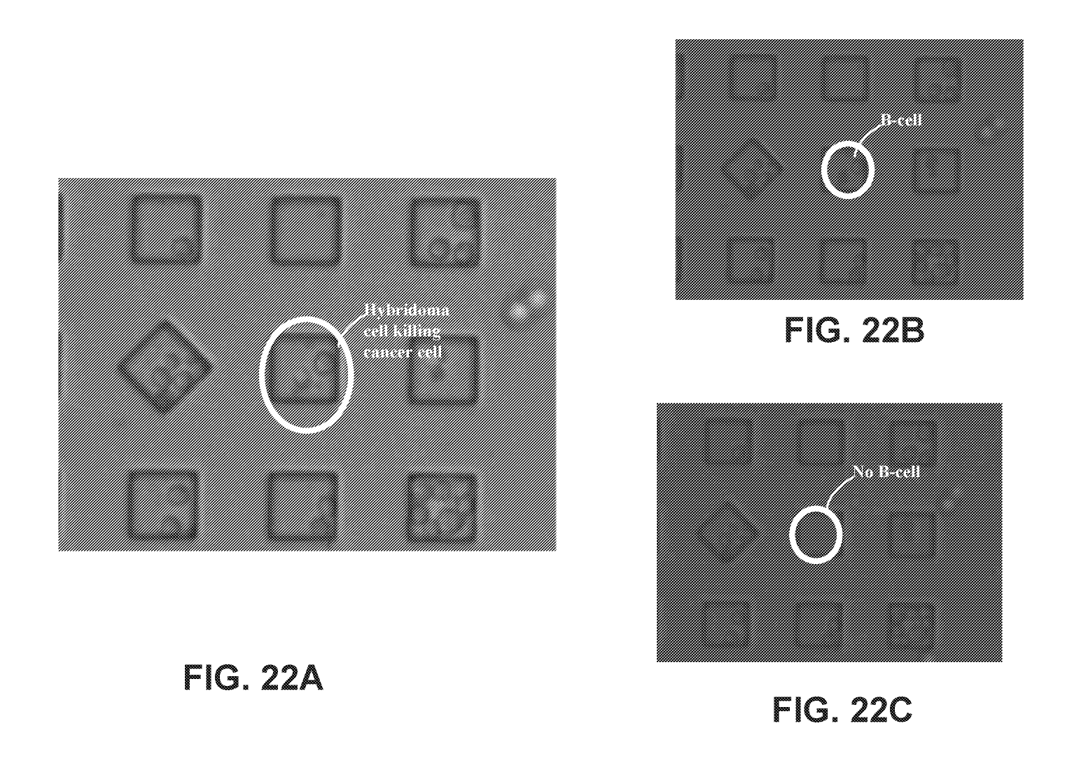

FIGS. 22A-C illustrates white blood cells identified as killing cancer cells, in accordance with various embodiments. Specifically, FIG. 22A illustrates a hybridoma cell detected as capable of killing cancer cells (e.g., a TNBC cell) in a nanowell array. FIG. 22B-22C illustrate images of the B-cell (that is detect as able to kill the TNBC cell) as detected (FIG. 22B) and the well after the B-cell is isolated (FIG. 22C) using cell picking circuitry.

Although the embodiments illustrated by the various experimental embodiments describe identification of antigen-specific antibody producing white blood cells from a human, embodiments are not so limited. For example, in various embodiments, antigen-specific antibody producing white blood cells can be identified and used for treatment of other organisms, such as various vertebrates including dogs, cats, horses, livestock, birds, fish, etc. As may be appreciated, any organism which has blood (e.g., white blood cells) capable of producing antibodies as an immune (or other) response can be used to identify antigen-specific antibodies. The blood sample used is from the specific organism. Further, the antigens used as targets are not limited to those identified herein and can include a variety of antigens.

Terms to exemplify orientation, such as on top, onto, within, may be used herein to refer to relative positions of elements as shown in the figures. It should be understood that the terminology is used for notational convenience only and that in actual use the disclosed structures may be oriented different from the orientation shown in the figures. Thus, the terms should not be construed in a limiting manner.

Various embodiments are implemented in accordance with the underlying Provisional Application (Ser. No. 62/198,550), entitled “Rapid Isolation and Sequencing of Human Antibodies from a Primary B-cell”, filed Feb. 26, 2016, to which benefit is claimed and is fully incorporated herein by reference. For instance, embodiments herein and/or in the provisional application (including the appendices therein) may be combined in varying degrees (including wholly). Reference may also be made to the experimental teachings and underlying references provided in the underlying provisional application. Embodiments discussed in the Provisional Application are not intended, in any way, to be limiting to the overall technical disclosure, or to any part of the claimed invention unless specifically noted.

As illustrated, various modules and/or other circuit-based building blocks (shown in the immediately preceding figure) may be implemented to carry out one or more of the operations and activities described herein, and/or shown in the block-diagram-type figures. In such contexts, these modules and/or building blocks represent circuits that carry out one or more of these or related operations/activities. For example, in certain of the embodiments discussed above, one or more modules and/or blocks are discrete logic circuits or programmable logic circuits configured for implementing these operations/activities, as in the circuit modules/blocks (e.g., the cell picking circuitry, processing circuitry, optic scanner, and fluorescent microscope) shown above. In certain embodiments, the programmable circuit is one or more computer circuits programmed to execute a set (or sets) of instructions (and/or configuration data). The instructions (and/or configuration data) can be in the form of firmware or software stored in and accessible from a memory (circuit). As an example, first and second modules/blocks include a combination of a CPU hardware-based circuit and a set of instructions in the form of firmware, where the first module/block includes a first CPU hardware circuit with one set of instructions and the second module/block includes a second CPU hardware circuit with another set of instructions.

Various embodiments described above, and discussed provisional application may be implemented together and/or in other manners. One or more of the items depicted in the present disclosure and in the underlying provisional application can also be implemented separately or in a more integrated manner, or removed and/or rendered as inoperable in certain cases, as is useful in accordance with particular applications. For example, the particular structures illustrated as shown and discussed may be replaced with other structures and/or combined together in the same apparatus. As another example, the methods illustrated by FIGS. 2 and 3 and can be implemented using the apparatus illustrated by FIG. 1. Further, the methods described by FIGS. 2-4 can be implemented together, separately, and/or using various combinations of the steps described there in. In view of the description herein, those skilled in the art will recognize that many changes may be made thereto without departing from the spirit and scope of the present disclosure.

1. A method comprising:
exposing immobilized white blood cells from a blood sample to an antigen; and
scanning the immobilized white blood cells and, therefrom, identify and isolate white blood cells from among the immobilized white blood cells that produce an antibody (Ab) in response to the exposure to the antigen.

2. The method of claim 1, further including scanning a whole white blood cell complement of the blood sample at a rate of 1 million to 25 million cells per minute.

3. The method of claim 1, further including immobilizing the white blood cells on a substrate while maintaining cell viability and exposing the immobilized white blood cells to the antigen by treating the white blood cells with the antigen which is labeled.

4. The method of claim 1, further including isolating the white blood cells individually using cell picking circuitry by identifying antigens bound to an antibody on a surface of or near white blood cells.

5. The method of claim 1, further including assessing efficacy of the produced Abs.

6. The method of claim 1, further including assessing an ability of the produced Abs to neutralize target cells associated with the antigen.

7. The method of claim 1, further including cloning at least one of the produced Abs and using the cloned Ab as at least one selected from the group consisting of: a diagnostic agent, a sensor, a therapeutic agent, and a combination thereof.

8. A method comprising:
causing a substrate coated with an antigen to contact a multiple-well array and thereby exposing a plurality of white blood cells to the antigen, each well of the multiple-well array including an individual white blood cell among the plurality of white blood cells;
causing each well of the multiple-well array to include an individual target cell; and
scanning the substrate and the wells of the multiple-well array and, therefrom, assessing an efficacy and cell function of the plurality of white blood cells.

9. The method of claim 8, further including depositing the white blood cells and the target cells into each well of the multiple-well array and co-culturing the white blood cells with the target cells while maintaining cell viability, the multiple-well array being a nanowell array.

10. The method of claim 9, further including isolating white blood cells among the plurality of white blood cells using cell picking circuitry, the isolated white blood cells identified as producing antigen-specific diagnostic and therapeutic antibodies.

11. The method of claim 9, further including forming an immuno-sandwich by:
incubating the substrate coated with the antigen in contact with the multiple-well array; and
treating the substrate with a labeled anti-human antibody.

12. The method of claim 9, further including labeling the plurality of white blood cells with at least one fluorescent label and the target cells with a different fluorescent label.

13. The method of claim 9, further including identifying and isolating white blood cells among the plurality of white blood cells that produce antigen-specific antibodies using data from the scanning of the substrate and the multiple-well array.

14. A method comprising:
on a substrate, immobilizing white blood cells from a blood sample;
exposing the immobilized white blood cells to an antigen;
identifying white blood cells from among the immobilized white blood cells that produce an antibody (Ab) in response to the exposure to the antigen by scanning the substrate using an optic scanner;
isolating the identified white blood cells from the substrate;
exposing a plurality of white blood cells, including the isolated white blood cells, to the antigen by causing an additional substrate coated with the antigen to contact a multiple-well array, each well of the multiple-well array including an individual white blood cell among the plurality of white blood cells;
exposing the plurality of white blood cells to target cells by causing each well of the multiple-well array to include an individual target cell; and
assessing an efficacy and cell function of the plurality of white blood cells by scanning the additional substrate and the wells of the multiple-well array using the optic scanner and a fluorescent microscope.

15. The method of claim 14, further including identifying and isolating, responsive to the assessment, white blood cells among the plurality of white blood cells that produce antigen-specific therapeutic and/or diagnostic antibodies.

16. The method of claim 14, wherein the assessment includes assessing an antigen-specific binding affinity of antibodies produced by the white blood cells and identifying antibodies that neutralize the target cells in wells of the multiple-well array.

17. The method of claim 16, further including cloning at least one of the produced antibodies and using the cloned antibody for use as a diagnostic or therapeutic agent.

18. The method of claim 14, further including treating the additional substrate with a labeled anti-human antibody and assessing an efficacy of the white blood cells by scanning the additional substrate using the optic scanner and, therefrom, identifying antibodies present on the additional substrate that are bound to the antigen.

19. An apparatus comprising:
an optic scanner, a light source, and imaging circuitry, the optic scanner configured to scan white blood cells from a blood sample and, therefrom, identify white blood cells that produce an antibody responsive to exposure to an antigen;
a fluorescent microscope configured to scan the white blood cells and, therefrom, verify the produced antibodies and assess an efficacy of the produced antibodies; and
cell picking circuitry configured to isolate antibodies responsive to the verification and the assessment.

20. The apparatus of claim 19, further including a nanowell array having the white blood cells contained with wells, each well including an individual white blood cell, where the fluorescent microscope is configured to image the nanowell array to analyze cell functionality of the white blood cells.
